(19) United States
(12) Patent Application Publication
(10) Pub. No.: US 2014/0294813 A1
(43) Pub. Date: Oct. 2, 2014

(54) TNF Binding Proteins

(71) Applicant: AbbVie, Inc., North Chicago, IL (US)

(72) Inventors: Tariq Ghayur, Holliston, MA (US); Dominic Ambrosi, Worcester, MA (US); Arun B. Deora, Framingham, MA (US); Subramanya Hegde, Shrewsbury, MA (US)

(73) Assignee: AbbVie, Inc., North Chicago, IL (US)

(21) Appl. No.: 14/210,703

(22) Filed: Mar. 14, 2014

Related U.S. Application Data
(60) Provisional application No. 61/788,113, filed on Mar. 15, 2013.

(51) Int. Cl. C07K 16/22

(52) U.S. Cl. CPC C07K 16/22
USPC 424/133.1; 530/387.3; 536/23.53; 435/320.1; 435/328

(57) ABSTRACT

Provided are TNF binding proteins and methods of treatment using the same. Also provided are nucleic acids encoding the binding proteins and recombinant expression vectors and host cells for making such binding proteins.

### RELATED APPLICATIONS

This application claims priority to U.S. Provisional Application Ser. No. 61/788,113, filed Mar. 15, 2013, which is incorporated herein by reference in its entirety.

This application is also related to U.S. Provisional Application Ser. No. 61/755,288, filed Jan. 22, 2013, U.S. Provisional Application Ser. No. 61/746,616, filed Dec. 28, 2012, and U.S. Provisional Application Ser. No. 61/746,617, filed Dec. 28, 2012, which are each incorporated herein by reference in their entireties.

### BACKGROUND

The use of therapeutic tissue necrosis factor alpha (TNFalpha) binding proteins, such as infliximab, adalimumab, etanercept, golimumab, and certolizumab has revolutionized the treatment of many chronic inflammatory diseases, including inflammatory bowel disease (IBD), ankylosing spondylitis, multiple sclerosis, psoriasis and rheumatoid arthritis (RA). Despite their success in improving the quality of life of patients, long-term treatment with therapeutic TNF binding proteins can elicit strong immunogenic responses that result in the development of anti-drug antibodies (ADA). Such ADA responses can impact both the safety and pharmacokinetics of therapeutic TNF binding proteins, which, in turn, can affect the utility and efficacy of these drugs. Accordingly, there is a need in the art for novel TNF binding proteins for use as therapeutics, which are less immunogenic in patients

### SUMMARY

The present disclosure provides novel TNF binding proteins and methods of treatment using the same. Also provided are nucleic acids encoding the binding proteins and recombinant expression vectors and host cells for making such binding proteins. The present disclosure is based, at least in part, on the discovery that bivalent TNF binding proteins (e.g., anti-TNF monoclonal antibodies) can bind to TNF on the cell surface of antigen presenting cells and become internalized. The binding proteins disclosed herein generally exhibit monovalent binding to cell-surface TNF alpha (i.e, each binding protein is only able to bind to one TNF molecule on the surface of an antigen presenting).

Accordingly, in one aspect, the present disclosure provides a binding protein that specifically binds to human TNF, wherein the binding protein comprises an antibody variable region and Fc region, and wherein and the binding protein exhibits an amount of cellular internalization upon binding to cell surface human TNF that is less than the amount of cellular internalization exhibited by an anti-human TNF reference antibody (e.g., infliximab, adalimumab, or golimumab). In certain embodiments, the binding protein binds monovalently to cell surface human TNF on antigen presenting cells.

In certain embodiments, the binding protein comprises a first polypeptide chain and a second polypeptide chain,

wherein the first polypeptide chain comprises VDH-(X1)n-C—Y1, wherein
VDH is a heavy chain variable domain,
X1 is a linker with the proviso that it is not CH1,
C is a CH1 domain,
Y1 is an Fc region,
n is 0 or 1;
and
wherein the second polypeptide chains comprises VDL-(X3)m-C, wherein
VDL is a light chain variable domain,
X3 is a linker with the proviso that it is not CH1,

### C is a CL1,

m is 0 or 1;

wherein X2 comprises at least one mutation that inhibits homodimerization of Y1. In one particular embodiment, Y1 comprises an amino acid sequence selected from the group set forth in Table 3. In one particular embodiment, X1 and/or X3 comprises an amino acid sequence set forth in Table 1. In one particular embodiment, VDH comprises the heavy chain CDRs or complete VH domain amino acid sequence of infliximab, adalimumab, certolizumab pegol, or golimumab. In one particular embodiment, VDL comprises the light chain CDRs or complete VL domain amino acid sequence of infliximab, adalimumab, certolizumab pegol, or golimumab.

In certain embodiments, the binding protein comprises a first polypeptide chain and a second polypeptide chain, wherein the first polypeptide chain comprises VDH1-(X1)n-VDH2-X2-(X3)m-Y1, wherein:

VDH1 is a first heavy chain variable domain;

X1 is a linker with the proviso that X1 is not CH1;

VDH2 is a second heavy chain variable domain;

X2 is CH1;

X3 is a linker;

Y1 is an Fc region;

n is 0 or 1, m is 0 or 1;

and
wherein the second polypeptide chain comprises VDL1-(X4)m-VDL2-X5, wherein:

VDL1 is a first light chain variable domain;

X4 is a linker with the proviso that X4 is not CH1;

VDL2 is a second light chain variable domain;

X5 is CL1;

m is 0 or 1,

wherein Y1 comprises at least one mutation that inhibits homodimerization of Y1. In one particular embodiment, X1, X2, and/or X4 comprises an amino acid sequence set forth in Table 1. In one particular embodiment, Y1 comprises an amino acid sequence set forth in Table 3. In one particular embodiment, VDH1 and/or VDH2 comprises the heavy chain CDRs or complete VH domain amino acid sequence of infliximab, adalimumab, certolizumab pegol, or golimumab. In one particular embodiment, VDL1 and/or VDL2 comprises the light chain CDRs or complete VL domain amino acid sequence of infliximab, adalimumab, certolizumab pegol, or golimumab.

In certain embodiments, the binding protein comprises four polypeptide chains, wherein two of said four polypeptide chains comprise VDH-(X1)n-C—Y1, wherein

VDH is a heavy chain variable domain,
X1 is a linker with the proviso that it is not CH1,
C is a CH1 domain,
Y1 is an Fc region,
n is 0 or 1;
and
wherein two of said four polypeptide chains comprise VDL-(X2)m-X3, wherein
VDL is a light chain variable domain,
X2 is a linker with the proviso that it is not CH1,
X3 is a CL domain,
m is 0 or 1;

wherein at least one of said four polypeptide chains comprises a mutation, said mutation being located in the variable domain, wherein said mutation inhibits the targeted binding between the specific antigen and the mutant binding domain. In one particular embodiment, Y1 comprises a mutation that enhances heterodimerization. In one particular embodiment, Y1 comprises an amino acid sequence set forth in Table 3. In one particular embodiment, X1 and/or X2 comprises an amino acid sequence set forth Table 1. In one particular embodiment, VDH comprises the heavy chain CDRs or complete VH domain amino acid sequence of infliximab, adalimumab, certolizumab pegol, or golimumab. In one particular embodiment, VDL comprises the light chain CDRs or complete VL domain amino acid sequence of infliximab, adalimumab, certolizumab pegol, or golimumab.

In certain embodiments, the binding protein comprises four polypeptide chains, wherein two of said four polypeptide chains comprise VDH1-(X1)n-VDH2-C—Y1, wherein

VDH1 is a first heavy chain variable domain,
VDH2 is a second heavy chain variable domain,
C is a heavy chain constant domain,
X1 is a linker with the proviso that it is not CH1,
Y1 is an Fc region,
n is 0 or 1;
and
wherein two of said four polypeptide chains comprise VDL1-(X2)m-VDL2-X3, wherein
VDL1 is a first light chain variable domain,
VDL2 is a second light chain variable domain,
X2 is a linker with the proviso that it is not CH1,
X3 is a CL domain,
m is 0 or 1;

wherein at least one of said four polypeptide chains comprises a mutation, said mutation being located in the first variable domain or the second variable domain, wherein said mutation inhibits the targeted binding between the specific antigen and the mutant binding domain. In one particular embodiment, the mutation is located in VDH1 and/or VDH2. In one particular embodiment, the mutation is located in VDL1 and/or VDL2. In one particular embodiment, Y1 comprises a mutation that enhances heterodimerization. In one particular embodiment, Y1 comprises an amino acid sequence set forth in Table 3. In one particular embodiment, X1 and/or X2 comprises and amino acid sequence set forth in Table 1. In one particular embodiment, VDH1 and/or VDH2 comprises the heavy chain CDRs or complete VH domain amino acid sequence of infliximab, adalimumab, certolizumab pegol, or golimumab. In one particular embodiment, VDL1 and/or VDL2 comprises the light chain CDRs or complete VL domain amino acid sequence of infliximab, adalimumab, certolizumab pegol, or golimumab.

In certain embodiments, the binding protein comprises a first polypeptide chain and a second polypeptide chain, said first polypeptide chain comprising VDH-(X1)n-X2-(X3)m-Y1, wherein:

VDH is a heavy chain variable domain;

X1 is a linker with the proviso that X1 is not CH1;

X2 is CH1;

X3 is a linker;

Y1 is an F region;

n is 0 or 1, m is 0 or 1;

and said second polypeptide comprising VDL-(X4)n-X5-(X6)m-Y2, wherein:

VDL is a light chain variable domain;

X4 is a linker with the proviso that X4 is not CH1;

X5 is CL1;

X6 is a linker;

Y2 is an F region;

n is 0 or 1, m is 0 or 1; wherein Y1 and Y2 each comprises a mutation, wherein the mutations on Y1 and Y2 enhance the interaction between Y1 and Y2. In one particular embodiment, Y1 and/or Y2 comprises an amino acid sequence set forth in Table 3. In one particular embodiment, X1, X3, X4, and/or X6 comprises and amino acid sequence set forth in Table 1. In one particular embodiment, VDH comprises the heavy chain CDRs or complete VH domain amino acid sequence of infliximab, adalimumab, certolizumab pegol, or golimumab. In one particular embodiment, VDL comprises the light chain CDRs or complete VL domain amino acid sequence of infliximab, adalimumab, certolizumab pegol, or golimumab.

In certain embodiments, the binding protein comprises a first polypeptide chain and a second polypeptide chain, said first polypeptide chain comprising VDH1-(X1)n-VDH2-X2-(X3)m-Y1, wherein:

VDH1 is a first heavy chain variable domain;

X1 is a linker with the proviso that X1 is not CH1;

VDH2 is a second heavy chain variable domain;

X2 is CH1;

X3 is a linker;

Y1 is an F region;

n is 0 or 1, m is 0 or 1;

and said second polypeptide comprising VDL1-(X4)n-VDL2-X5-(X6)m-Y2, wherein:

VDL1 is a first light chain variable domain;

X4 is a linker with the proviso that X4 is not CH1;

VDL2 is a second light chain variable domain;

X5 is CL1;

X6 is a linker;

Y2 is an F region;

n is 0 or 1, m is 0 or 1, wherein Y1 and Y2 each comprises a mutation, wherein the mutations on Y1 and Y2 enhance heterodimerization between Y1 and Y2. In one particular embodiment, Y1 and/or Y2 comprises an amino acid sequence set forth in Table 3. In one particular embodiment, X1 and/or X3, comprises and amino acid sequence set forth in Table 1. In one particular embodiment, VDH1 and/or VDH2 comprises the heavy chain CDRs or complete VH domain amino acid sequence of infliximab, adalimumab, certolizumab pegol, or golimumab. In one particular embodiment, VDL1 and/or VDL2 comprises the light chain CDRs or complete VL domain amino acid sequence of infliximab, adalimumab, certolizumab pegol, or golimumab.

In certain embodiments, the binding protein comprises a first, second, third and fourth polypeptide chains,

wherein said first polypeptide chain comprises VD1-(X1)n-VD2-CH—(X2)n, wherein VD1 is a first heavy chain variable domain, VD2 is a second heavy chain variable domain, C is a CH1 domain, X1 is a linker with the proviso that it is not a constant domain, and X2 is an Fc region; wherein said second polypeptide chain comprises VD1-(X1)n-VD2-CL-(X2)n, wherein VD1 is a first light chain variable domain, VD2 is a second light chain variable domain, CL is a light chain constant domain, X1 is a linker with the proviso that it is not a constant domain, and X2 does not comprise an Fc region; wherein said third polypeptide chain comprises VD3-(X3)n-VD4-CL-(X4)n, wherein VD3 is a third heavy chain variable domain, VD4 is a fourth heavy chain variable domain, CL is a light chain constant domain, X3 is a linker with the proviso that it is not a constant domain, and X4 is an Fc region; wherein said fourth polypeptide chain comprises VD3-(X3)n-VD4-CH—(X4)n, wherein VD3 is a third light chain variable domain, VD4 is a fourth light chain variable domain, CH is CH1 domain, X3 is a linker with the proviso that it is not a constant domain, and X4 does not comprise an Fc region; wherein n is 0 or 1, and wherein the VD 1 domains on the first and second polypeptide chains form one functional binding site for a first antigen, the VD2 domains on the first and second polypeptide chains form one functional binding site for a second antigen, the VD3 domains on the third and fourth polypeptide chains form one functional binding site for a third antigen, and the VD4 domains on the third and fourth polypeptide chains form one functional binding site for forth antigen. In one particular embodiment, at least one of the first, second, third or forth antigens is human TNF. In one particular embodiment, X2 and/or X4 comprises at least one mutation that enhances heterodimerization of X2 and X4. In one particular embodiment, X2 and/or X4 comprises an amino acid sequence set forth in Table 3. In one particular embodiment, X1 and/or X3, comprises and amino acid sequence set forth in Table 1. In one particular embodiment, VD1, VD2, VD3, and/or VD4 comprise the heavy chain CDRs, the light chain CDRs, the complete VH domain, or the complete VL domain amino acid sequence of infliximab, adalimumab, certolizumab pegol, or golimumab.

In certain embodiments, the binding protein comprises a polypeptide chain, wherein the polypeptide chain comprises RD1-(X)n-VDH-C—Y or VDH-(X)n-RD1-C—Y, wherein

RD1 comprises a ligand-binding domain of a receptor;
VDH is a heavy chain variable domain;
C is a heavy chain constant domain;
X is a linker with the proviso that it is not CH1;
Y is an Fc region; and
n is 0 or 1.

In one particular embodiment, RD1 comprises a receptor that binds to human TNF. In one particular embodiment, RD1 comprises a TNF receptor binding portion of etanercept. In one particular embodiment, VDH comprises the heavy chain CDRs, or the complete VH domain acid sequence of infliximab, adalimumab, certolizumab pegol, or golimumab.

In another aspect, the present disclosure provides a composition comprising a binding polypeptide of any one of the preceding claims and a pharmaceutically acceptable carrier or excipient.

In another aspect, the present disclosure provides a method of treating a TNF-associated disorder in a subject in need thereof, comprising administering to the subject an effective amount of the compositions disclosed herein.

In another aspect, the present disclosure provides an isolated polynucleotide encoding a binding polypeptide disclosed herein.

In another aspect, the present disclosure provides a vector comprising a polynucleotide disclosed herein.

In another aspect, the present disclosure provides a host cell comprising a polynucleotide or vector disclosed herein.

### BRIEF DESCRIPTION OF THE DRAWINGS

FIG. 1 depicts the results of experiments measuring the surface expression of TNFalpha on peripheral blood monocytes stimulated with LPS.

FIG. 2 depicts the results of experiments measuring the surface expression of TNFalpha on peripheral blood monocytes and T cells stimulated with LPS.

FIG. 3 depicts the results of experiments measuring the internalization of an anti-TNFalpha antibody by peripheral blood mononuclear cells stimulated with LPS.

FIG. 4 depicts the results of experiments measuring the surface expression of TNFalpha on LPS treated human monocytes.

FIG. 5 depicts the results of experiments measuring the surface expression of TNFalpha on peripheral blood monocytes stimulated with GM-CSF and LPS.

FIG. 6 depicts the results of experiments measuring the surface expression of TNFalpha on cells stimulated with LPS.

FIG. 7 depicts the results of experiments measuring (A) the surface expression of TNFalpha on human monocyte derived dendritic cells stimulated with LPS, and (B) the levels of soluble TNFalpha in culture medium.

FIG. 8 depicts the results of experiments measuring the internalization of an anti-TNFalpha antibody by monocytes derived dendritic cells stimulated with LPS using (A) a pH-sensitive dye and (B) nuclear stain.

FIG. 9 depicts the results of experiments measuring the internalization kinetics of an anti-TNFalpha antibody by peripheral blood mononuclear cells stimulated with LPS.

FIG. 10 depicts an exemplary (A) half-body monovalent anti-TNF antibody and (B) half-body DVD-Ig molecules, as disclosed herein.

FIG. 11 depicts exemplary AbbmAb (A) monovalent anti-TNF antibody or (B) monovalent DVD-Ig molecules, as disclosed herein.

FIG. 12 depicts exemplary (A) monovalent anti-TNF immunoglubins (M-body) antibody based molecules and (B) M-body DVD-Ig based molecules, as disclosed herein.

FIG. 13 depicts exemplary (A) multi-variable, monovalent anti-TNF poly-Ig antibody based molecules and (B) multi-variable, monovalent anti-TNF poly-Ig DVD-Ig based molecules, as disclosed herein.

### DETAILED DESCRIPTION

The present disclosure provides novel TNF binding proteins and methods of treatment using the same. Also provided are nucleic acids encoding the binding proteins and recombinant expression vectors and host cells for making such binding proteins. The present disclosure is based, at least in part, on the discovery that bivalent TNF binding proteins (e.g., anti-TNF monoclonal antibodies) can bind to TNF on the cell surface of antigen presenting cells and become internalized. The binding proteins disclosed herein are generally monovalent with regard to cell surface TNF binding (i.e, each binding protein is only able to bind to one TNF molecule on the surface of an antigen presenting).

### I. DEFINITIONS

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well known and commonly used in the art.

In order that the present invention may be more readily understood, certain terms are first defined.

As used herein, the term “monobody DVD” or “mDVD” refers to monovalent DVD-Ig molecules as described in U.S. Provisional Application Ser. No. 61/755,288, which is incorporated herein by reference in its entirety.

As used herein, the term “polyvalent DVD” or “pDVD” refers to polyvalent DVD-Ig molecules as described in U.S. Provisional Application Ser. No. 61/746,616, which is incorporated herein by reference in its entirety.

As used herein, the term “receptor DVD” or “rDVD” refers to receptor-DVD-Ig molecules as described in U.S. Provisional Application Ser. No. 61/746,617, which is incorporated herein by reference in its entirety.

As used herein, the term “infliximab” refers to the anti-TNF antibody marketed as REMICADE™, having Chemical Abstracts Service (CAS) designation 170277-31-3.

As used herein, the term “golimumab” refers to the anti-TNF antibody marketed as SIMPONI™, having Chemical Abstracts Service (CAS) designation 476181-74-5.

As used herein, the term “certolizumab” refers to the anti-TNF antibody marketed as CIMZIA™, having Chemical Abstracts Service (CAS) designation 428863-50-7.

As used herein, the term “adalimumab” refers to the anti-TNF antibody marketed as HUMIRA™, having Chemical Abstracts Service (CAS) designation 331731-18-1.

As used herein, the term “infliximab” refers to the anti-TNF immunoadhesin marketed as ENBREL™, having Chemical Abstracts Service (CAS) designation 1094-08-2.

The term “human TNF-alpha”, as used herein, is intended to refer to a human cytokine that exists as a 17 kD secreted form and a 26 kD membrane associated form, the biologically active form of which is composed of a trimer of noncovalently bound 17 kD molecules. The structure of humanTNF-alpha is described further in, for example, Pennica, D., et al. (1984) Nature 312:724-729; Davis, J. M., et al. (1987) Biochemistry 26:1322-1326; and Jones, E. Y., et al. (1989) Nature 338:225-228. The term human TNF-alpha is intended to include recombinant human TNF-alpha, which can be prepared by standard recombinant expression methods or purchased commercially (R & D Systems, Catalog No. 210-TA, Minneapolis, Minn.).

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (Winter, et al. U.S. Pat. Nos. 5,648,260; 5,624,821). The Fc portion of an antibody mediates several important effector functions e.g. cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to Fc.gamma.Rs and complement Clq, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered. The dimerization of two identical heavy chains of an immunoglobulin is mediated by the dimerization of CH3 domains and is stabilized by the disulfide bonds within the hinge region (Huber et al. Nature; 264: 415-20; Thies et al 1999 J Mol Biol; 293: 67-79.). Mutation of cysteine residues within the hinge regions to prevent heavy chain-heavy chain disulfide bonds will destabilize dimeration of CH3 domains. Residues responsible for CH3 dimerization have been identified (Dall'Acqua 1998 Biochemistry 37: 9266-73.). Therefore, it is possible to generate a monovalent half-Ig. Interestingly, these monovalent half Ig molecules have been found in nature for both IgG and IgA subclasses (Seligman 1978 Ann Immunol 129: 855-70; Biewenga et al 1983 Clin Exp Immunol 51: 395-400). The stoichiometry of FcRn: Ig Fc region has been determined to be 2:1 (West et al 2000 Biochemistry 39: 9698-708), and half Fc is sufficient for mediating FcRn binding (Kim et al 1994 Eur J Immunol; 24: 542-548.). Mutations to disrupt the dimerization of CH3 domain may not have greater adverse effect on its FcRn binding as the residues important for CH3 dimerization are located on the inner interface of CH3 b sheet structure, whereas the region responsible for FcRn binding is located on the outside interface of CH2-CH3 domains. However the half Ig molecule may have certain advantage in tissue penetration due to its smaller size than that of a regular antibody. In one embodiment at least one amino acid residue is replaced in the constant region of the binding protein of the invention, for example the Fc region, such that the dimerization of the heavy chains is disrupted, resulting in half DVD Ig molecules.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′).sub.2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5). In addition single chain antibodies also include “linear antibodies” comprising a pair of tandem Fv segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

As used herein, the terms “VH domain” and “VL domain” refer to single antibody variable heavy and light domains, respectively, comprising FR (Framework Regions) 1, 2, 3 and 4 and CDR (Complementary Determinant Regions) 1, 2 and 3 (see Kabat et al. (1991) Sequences of Proteins of Immunological Interest. (NIH Publication No. 91-3242, Bethesda).

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat, based on sequence comparisons.

As used herein the term “framework (FR) amino acid residues” refers to those amino acids in the framework region of an immunogobulin chain. The term “framework region” or “FR region” as used herein, includes the amino acid residues that are part of the variable region, but are not part of the CDRs (e.g., using the Kabat definition of CDRs).

As used herein, the term “specifically binds to” refers to the ability of a binding polypeptide to bind to an antigen with an Kd of at least about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or bind to an antigen with an affinity that is at least two-fold greater than its affinity for a nonspecific antigen. It shall be understood, however, that the binding polypeptide are capable of specifically binding to two or more antigens which are related in sequence. For example, the binding polypeptides of the invention can specifically bind to both human and a non-human (e.g., mouse or non-human primate) orthologos of an antigen.

The term “Polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “linker” is used to denote polypeptides comprising two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). Preferred linkers include, but are not limited to, the amino acid linkers set forth in Table 7 herein.

The term “Kₒₙ”, as used herein, is intended to refer to the on rate constant for association of an antibody to the antigen to form the antibody/antigen complex as is known in the art.

The term “K_{off}”, as used herein, is intended to refer to the off rate constant for dissociation of an antibody from the antibody/antigen complex as is known in the art.

The term “Kd”, as used herein, is intended to refer to the dissociation constant of a particular antibody-antigen interaction as is known in the art.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred eukaryotic cells include protist, fungal, plant and animal cells. Most preferably host cells include but are not limited to the prokaryotic cell line E. Coli; mammalian cell lines CHO, HEK 293 and COS; the insect cell line Sf9; and the fungal cell Saccharomyces cerevisiae.

### II. IMPROVED TNF BINDING PROTEINS

In one aspect the invention provides novel TNF binding proteins. These binding proteins exhibit monovalent binding to TNF alpha on the surface of a cell (e.g., an antigen presenting cell), i.e, each binding protein is only able to bind to one TNF molecule on the surface of an antigen presenting). In certain embodiments, the binding proteins disclosed herein binds to human TNF, wherein the binding protein exhibits a reduced of cellular internalization upon binding to cell surface TNF compared to the cellular internalization exhibited by a reference antibody (e.g., infliximab, adalimumab, certolizumab pegol, or golimumab).

In certain embodiments, the TNF binding domains of known TNF binding agents are reformatted to produce the novel TNF binding proteins disclosed herein. The TNF binding domains of any TNF binding agents can be employed including. In certain embodiments, the variable domains (or CDRs thereof) of the anti-TNF antibodies infliximab, adalimumab, certolizumab pegol, and/or golimumab are employed. In certain embodiments, the TNF binding domain of etanercept is employed. In certain embodiments, one of more of the variable domain amino an amino acid set forth in Table 2 are employed.

Any binding protein format that achieves monovalent binding to cell surface TNF can be employed in the TNF binding proteins disclosed herein. In certain embodiments, the TNF binding proteins are “monobody DVD” or “mDVD” molecules, as described in U.S. Provisional Application Ser. No. 61/755,288, which is incorporated by reference herein in its entirety. In certain embodiments, the TNF binding proteins are “polyvalent DVD” or “pDVD” molecules described in U.S. Provisional Application Ser. No. 61/746,616, which is incorporated by reference herein in its entirety. In certain embodiments, the TNF binding proteins are “receptor DVD” or “rDVD” molecules, as described in U.S. Provisional Application Ser. No. 61/746,617, which is incorporated by reference herein in its entirety.

In certain embodiments, the TNF binding proteins are half antibody molecules comprising a first polypeptide chain and a second polypeptide chain,

wherein the first polypeptide chain comprises VDH-(X1)n-C—Y1, wherein
VDH is a heavy chain variable domain,
X1 is a linker with the proviso that it is not CH1,
C is a CH1 domain,
Y1 is an Fc region,
n is 0 or 1;
and wherein the second polypeptide chains comprises VDL-(X3)m-C, wherein
VDL is a light chain variable domain,
X3 is a linker with the proviso that it is not CH1,

### C is a CL1,

m is 0 or 1, wherein X2 comprises at least one mutation that inhibits dimerization of Y1.

In certain embodiments, the TNF binding proteins are half-DVD molecules comprising a first polypeptide chain and a second polypeptide chain, wherein the first polypeptide chain comprises VDH1-(X1)n-VDH2-X2-(X3)m-Y1, wherein:

VDH1 is a first heavy chain variable domain;

X1 is a linker with the proviso that X1 is not CH1;

VDH2 is a second heavy chain variable domain;

X2 is CH1;

X3 is a linker;

Y1 is an Fc region;

n is 0 or 1, m is 0 or 1;

and wherein the second polypeptide chain comprises VDL1-(X4)m-VDL2-X5, wherein:

VDL1 is a first light chain variable domain;

X4 is a linker with the proviso that X4 is not CH1;

VDL2 is a second light chain variable domain;

X5 is CL1;

m is 0 or 1, wherein Y1 comprises at least one mutation that inhibits homodimerization of Y1.

In certain embodiments, the TNF binding proteins are monobody molecules comprising four polypeptide chains, wherein two of said four polypeptide chains comprise VDH-(X1)n-C—

Y1, wherein
VDH is a heavy chain variable domain,
X1 is a linker with the proviso that it is not CH1,
C is a CH1 domain,
Y1 is an Fc region,
n is 0 or 1;
and wherein two of said four polypeptide chains comprise VDL-(X2)m-X3, wherein
VDL is a light chain variable domain,
X2 is a linker with the proviso that it is not CH1,
X3 is a CL domain,
m is 0 or 1, wherein at least one of said four polypeptide chains comprises a mutation, said mutation being located in the variable domain, wherein said mutation inhibits the targeted binding between the specific antigen and the mutant binding domain.

In certain embodiments, the TNF binding proteins are monobody molecules comprising four polypeptide chains, wherein two of said four polypeptide chains comprise VDH1-(X1)n-VDH2-C—Y1, wherein

VDH1 is a first heavy chain variable domain,
VDH2 is a second heavy chain variable domain,
C is a heavy chain constant domain,
X1 is a linker with the proviso that it is not CH1,
Y1 is an Fc region,
n is 0 or 1;
and wherein two of said four polypeptide chains comprise VDL1-(X2)m-VDL2-X3, wherein
VDL1 is a first light chain variable domain,
VDL2 is a second light chain variable domain,
X2 is a linker with the proviso that it is not CH1,
X3 is a CL domain,
m is 0 or 1, wherein at least one of said four polypeptide chains comprises a mutation, said mutation being located in the first variable domain or the second variable domain, wherein said mutation inhibits the targeted binding between the specific antigen and the mutant binding domain.

In certain embodiments, the TNF binding proteins are one-armed monobody molecules comprising a first polypeptide chain and a second polypeptide chain, said first polypeptide chain comprising VDH-(X1)n-X2-(X3)m-Y1, wherein:

VDH is a heavy chain variable domain;

X1 is a linker with the proviso that X1 is not CH1;

X2 is CH1;

X3 is a linker;

Y1 is an F region;

n is 0 or 1, m is 0 or 1;

and said second polypeptide comprising VDL-(X4)n-X5-(X6)m-Y2, wherein:

VDL is a light chain variable domain;

X4 is a linker with the proviso that X4 is not CH1;

X5 is CL1;

X6 is a linker;

Y2 is an F region;

n is 0 or 1, m is 0 or 1; wherein Y1 and Y2 each comprises a mutation, wherein the mutations on Y1 and Y2 enhance the interaction between Y1 and Y2.

In certain embodiments, the TNF binding proteins are one-armed monobody DVD-Ig molecules comprising a first polypeptide chain and a second polypeptide chain, said first polypeptide chain comprising VDH1-(X1)n-VDH2-X2-(X3)m-Y1, wherein:

VDH1 is a first heavy chain variable domain;

X1 is a linker with the proviso that X1 is not CH1;

VDH2 is a second heavy chain variable domain;

X2 is CH1;

X3 is a linker;

Y1 is an F region;

n is 0 or 1, m is 0 or 1;

and said second polypeptide comprising VDL1-(X4)n-VDL2-X5-(X6)m-Y2, wherein:

VDL1 is a first light chain variable domain;

X4 is a linker with the proviso that X4 is not CH1;

VDL2 is a second light chain variable domain;

X5 is CL1;

X6 is a linker;

Y2 is an F region;

n is 0 or 1, m is 0 or 1, wherein Y1 and Y2 each comprises a mutation, wherein the mutations on Y1 and Y2 enhance heterodimerization between Y1 and Y2.

In certain embodiments, the TNF binding proteins are polyvalent DVD-Ig molecules comprising first, second, third and fourth polypeptide chains,

wherein said first polypeptide chain comprises VD1-(X1)n-VD2-CH—(X2)n, wherein VD1 is a first heavy chain variable domain, VD2 is a second heavy chain variable domain, C is a CH1 domain, X1 is a linker with the proviso that it is not a constant domain, and X2 is an Fc region;
wherein said second polypeptide chain comprises VD1-(X1)n-VD2-CL-(X2)n, wherein VD1 is a first light chain variable domain, VD2 is a second light chain variable domain, CL is a light chain constant domain, X1 is a linker with the proviso that it is not a constant domain, and X2 does not comprise an Fc region; wherein said third polypeptide chain comprises VD3-(X3)n-VD4-CL-(X4)n, wherein VD3 is a third heavy chain variable domain, VD4 is a fourth heavy chain variable domain, CL is a light chain constant domain, X3 is a linker with the proviso that it is not a constant domain, and X4 is an Fc region; wherein said fourth polypeptide chain comprises VD3-(X3)n-VD4-CH—(X4)n, wherein VD3 is a third light chain variable domain, VD4 is a fourth light chain variable domain, CH is CH1 domain, X3 is a linker with the proviso that it is not a constant domain, and X4 does not comprise an Fc region; wherein n is 0 or 1, and wherein the VD 1 domains on the first and second polypeptide chains form one functional binding site for a first antigen, the VD2 domains on the first and second polypeptide chains form one functional binding site for a second antigen, the VD3 domains on the third and fourth polypeptide chains form one functional binding site for a third antigen, and the VD4 domains on the third and fourth polypeptide chains form one functional binding site for forth antigen.

In certain embodiments, the TNF binding proteins are receptor DVD (rDVD) molecules comprising a polypeptide chain, wherein the polypeptide chain comprises RD1-(X)n-VDH-C—Y or VDH-(X)n-RD1-C—Y, wherein

RD1 comprises a ligand-binding domain of a receptor;
VDH is a heavy chain variable domain;
C is a heavy chain constant domain;
X is a linker with the proviso that it is not CH1;
Y is an Fc region; and
n is 0 or 1.

Any amino acid linker can be used in the TNF binding proteins disclosed herein. In certain embodiments, the linker comprises amino an amino acid sequence selected from those set forth in Table 1.

Any Fc mutants can be used to achieve the half-molecules (e.g, half-antibodies and half-DVD-Ig), or heteromeric molecules (e.g., pDVD and mDVD) disclosed herein. In certain embodiments, the Fc mutants are selected from those set forth in Table 3.

TABLE 1
List of Linkers Used in Construction of
Monovalent TNF Binding Molecules

| SEQ ID NO | Linker Name | Sequence |
|---|---|---|
| | HG-short | ASTKGP |
| | LK-short | TVAAP |
| | LK-long | TVAAPSVFIFPP |
| | HG-long | ASTKGPSVFPLAP |
| | GS-H5 | GGGGSG |
| | GS-L5 | GGSGG |
| | QH | QEPKSSDKTHTSP |
| | N/A | AKTTPKLEEGEFSEAR |
| | N/A | AKTTPKLEEGEFSEARV |
| | N/A | AKTTPKLGG |
| | N/A | SAKTTPKLGG |
| | N/A | SAKTTP |
| | N/A | RADAAP |
| | N/A | RADAAPTVS |
| | N/A | RADAAAAGGPGS |
| | N/A | RADAAAA(G4S)4 |
| | N/A | SAKTTPKLEEGEFSEARV |
| | N/A | ADAAP |
| | N/A | ADAAPTVSIFPP |
| | N/A | TVAAP |
| | N/A | TVAAPSVFIFPP |
| | N/A | QPKAAP |
| | N/A | QPKAAPSVTLFPP |
| | N/A | AKTTPP |
| | N/A | AKTTPPSVTPLAP |
| | N/A | AKTTAP |
| | N/A | AKTTAPSVYPLAP |
| | N/A | ASTKGP |
| | N/A | ASTKGPSVFPLAP |
| | N/A | GGGGSGGGGSGGGGS |
| | N/A | GENKVEYAPALMALS |
| | N/A | GPAKELTPLKEAKVS |
| | N/A | GHEAAAVMQVQYPAS |
| | N/A | TVAAPSVFIFPPTVAAPSVFIFPP |
| | N/A | ASTKGPSVFPLAPASTKGPSVFPLAP |
| | G4S repeats | (GGGGS)N |
| | GS-H7 | GGGGSGG |
| | GS-H10 | GGGGSGGGGS |
| | GS-H13 | GGGGSGGGGSGGG |
| | HEH-7 | TPAPLPT |
| | HEH-13 | TPAPLPAPLPAPT |
| | HNG-9 | TSPPSPAPE |
| | HNG-12 | TSPPSPAPELLG |

TABLE 2
Examples of Anti-TNF Binding Molecules

| SEQ | | | |
|---|---|---|---|
| ID | | | Fc |
| NO | Name | Sequence | ID |
| | MAK1 | EVQLVQSGAEVKKPGASVKVSCKASGYTFNNY | Half |
| | 99.4 | GIIWVRQAPGQGLEWMGWINTYTGKPTYAQKF | body |
| | | QGRVTMTTDTSTSTAYMELSSLRSEDTAVYYC | |
| | | ARKLFNTVAVTDNAMDYWGQGTTVTVSS | |
| | MAK1 | DIQMTQSPSSLSASVGDRVTITCRASQDIENY | hCk |
| | 99.4 | LNWYQQKPGKAPKLLIYYTSRLQSGVPSRFSG | |
| | | SGSGTDFTLTISSLQPEDFATYFCQQGNTQPP | |
| | | TFGQGTKLEIKR | |
| | MAK1 | EVQLVESGGGLVQPGGSLRLSCAASGFTFSNY | Half |
| | 95.24 | GVEWVRQAPGKGLEWVSGIWADGSTHYADTVK | body |
| | | SRFTISRDNSKNTLYLQMNSLRAEDTAVYYCA | |
| | | REWQHGPVAYWGQGTLVTVSS | |
| | MAK1 | DIQMTQSPSSLSASVGDRVTITCKASQLVSSA | hCk |
| | 95.24 | VAWYQQKPGKAPKLLIYWASTLHTGVPSRFSG | |
| | | SGSGTDFTLTISSLQPEDFATYYCQQHYRTPF | |
| | | TFGQGTKLEIKR | |
| | MAK1 | EVQLVESGGGLVQPGGSLRLSCAASGFTFSNY | Half |
| | 99.21 | GVTWVRQAPGKGLEWVSMIWADSTHYASSVKG | body |
| | | RFTISRDNSKNTLYLQMNSLRAEDTAVYYCAR | |
| | | EWQHGPVAYWGQGTLVTVSS | |
| | MAK1 | DIQMTQSPSSLSASVGDRVTITCRASQLVSSA | hCk |
| | 99.21 | VAWYQQKPGKAPKLLIYWASARHTGVPSRFSG | |
| | | SGSGTDFTLTISSLQPEDFATYYCQQHYKTPF | |
| | | TFGQGTKLEIKR | |
| | 10F7 | EVQLVQSGAEVKKPGSSVKVSCKASGYTFTDY | Half |
| | M11 | EIHWVRQAPGQGLEWMGVNDPESGGTFYNQKF | body |
| | | DGRVTLTADESTSTAYMELSSLRSEDTAVYYC | |
| | | TRYSKWDSFDGMDYWGQGTTVTVSS | |
| | 10F7 | DIQMTQSPSSLSASVGDRVTITCRASSGIISY | hCk |
| | M11 | IDWFQQKPGKAPKRLIYATFDLASGVPSRFSG | |
| | | SGSGTDYTLTISSLQPEDFATYYCRQVGSYPE | |
| | | TFGQGTKLEIKR | |

TABLE 3
Sequence of Fc Variants for Producing
Monovalent Binding Proteins

| | | SEQ |
|---|---|---|
| | | ID |
| Name | Sequence | NO |
| Half | ASTKGPSVFPLAPSSKSTSGGTAALGCLVKD | |
| body | YFPEPVTVSWNSGALTSGVHTFPAVLQSSGL | |
| constant | YSLSSVVTVPSSSLGTQTYICNVNHKPSNTK | |
| region | VDKKVEPKSCDKTHTSPPSPAPELLGGPSVF | |
| | LFPPKPKDTLMISRTPEVTCVVVDVSHEDPE | |
| | VKFNWYVDGVEVHNAKTKPREEQYNSTYRVV | |
| | SVLTVLHQDWLNGKEYKCKVSNKALPAPIEK | |
| | TISKAKGQPREPQVYTLPPSREEMTKNQVSL | |
| | TCLVKGFYPSDIAVEWESNGQPENNYKTTPP | |
| | VLDSDGSFRLYSKLTVDKSRWQQGNVFSCSV | |
| | MHEALHNHYTQKSLSLSPGK | |
| AvvMab | VQCSGTTDKTHTCPPCPAPELLGGPSVFLFP | |
| CH2-CH3 | PKPKDTLMISRTPEVTCVVVDVSHEDPEVKF | |
| knob | NWYVDGVEVHNAKTKPREEQYNSTYRVVSVL | |
| | TVLHQDWLNGKEYKCKVSNKALPAPIEKTIS | |
| | KAKGQPREPQVYTLPPSREEMTKNQVSLWCL | |
| | VKGFYPSDIAVEWESNGQPENNYKTTPPVLD | |
| | SDGSFFLYSKLTVDKSRWQQGNVFSCSVMHE | |
| | ALHNHYTQKSLSLSPGK | |
| pCH123Kn | ASTKGPSVFPLAPSSKSTSGGTAALGCLVKD | |
| constant | YFPEPVTVSWNSGALTSGVHTFPAVLQSSGL | |
| region | YSLSSVVTVPSSSLGTQTYICNVNHKPSNTK | |
| (Knob) | VDKKVEPKSCDKTHTCPPCPAPELLGGPSVF | |
| | LFPPKPKDTLMISRTPEVTCVVVDVSHEDPE | |
| | VKFNWYVDGVEVHNAKTKPREEQYNSTYRVV | |
| | SVLTVLHQDWLNGKEYKCKVSNKALPAPIEK | |
| | TISKAKGQPREPQVYTLPPSREEMTKNQVSL | |
| | WCLVKGFYPSDIAVEWESNGQPENNYKTTPP | |
| | VLDSDGSFFLYSKLTVDKSRWQQGNVFSCSV | |
| | MHEALHNHYTQKSLSLSPGK | |
| pCH123h | ASTKGPSVFPLAPSSKSTSGGTAALGCLVKD | |
| constant | YFPEPVTVSWNSGALTSGVHTFPAVLQSSGL | |
| region | YSLSSVVTVPSSSLGTQTYICNVNHKPSNTK | |
| (hole) | VDKKVEPKSCDKTHTCPPCPAPELLGGPSVF | |
| | LFPPKPKDTLMISRTPEVTCVVVDVSHEDPE | |
| | VVKFNWYVDGVEVHNAKTKPREEQYNSTYRV | |
| | SVLTVLHQDWLNGKEYKCKVSNKALPAPIEK | |
| | TISKAKGQPREPQVYTLPPSREEMTKNQVSL | |
| | SCAVKGFYPSDIAVEWESNGQPENNYKTTPP | |
| | VLDSDGSFFLVSKLTVDKSRWQQGNVFSCSV | |
| | MHEALHNHYTQKSLSLSPGK | |
| hCk | TVAAPSVFIFPPSDEQLKSGTASVVCLLNNF | |
| | YPREAKVQWKVDNALQSGNSQESVTEQDSKD | |
| | STYSLSSTLTLSKADYEKHKVYACEVTHQGL | |
| | SSPVTKSFNRGEC | |

### III. ENGINEERED TNF BINDING PROTEINS

In certain preferred embodiments, the TNF binding proteins produced using the methods and compositions disclosed herein exhibit improved properties (e.g., affinity or stability) with respect to a corresponding parental reference binding protein. For example, the engineered binding protein may dissociate from its target antigen with a k_{off} rate constant of about 0.1 s⁻¹ or less, as determined by surface plasmon resonance, or inhibit the activity of the target antigen with an IC₅₀ of about 1×10⁻⁶M or less. Alternatively, the binding protein may dissociate from the target antigen with a k_{off} rate constant of about 1×10⁻² s⁻¹ or less, as determined by surface plasmon resonance, or may inhibit activity of the target antigen with an IC₅₀ of about 1×10⁻⁷M or less. Alternatively, the binding protein may dissociate from the target with a k_{off} rate constant of about 1×10⁻³ s⁻¹ or less, as determined by surface plasmon resonance, or may inhibit the target with an IC₅₀ of about 1×10⁻⁸M or less. Alternatively, binding protein may dissociate from the target with a k_{off} rate constant of about 1×10⁻⁴ s⁻¹ or less, as determined by surface plasmon resonance, or may inhibit its activity with an IC₅₀ of about 1×10⁻⁹M or less. Alternatively, binding protein may dissociate from the target with a k_{off} rate constant of about 1×10⁻⁵ s⁻¹ or less, as determined by surface plasmon resonance, or inhibit its activity with an IC₅₀ of about 1×10⁻¹⁰M or less. Alternatively, binding protein may dissociate from the target with a k_{off} rate constant of about 1×10⁻⁵ s⁻¹ or less, as determined by surface plasmon resonance, or may inhibit its activity with an IC₅₀ of about 1×10⁻¹¹M or less.

In certain embodiments, the engineered binding protein comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the binding protein can comprise a light chain constant region, either a kappa light chain constant region or a lambda light chain constant region. The binding protein comprises a kappa light chain constant region. Alternatively, the binding protein portion can be, for example, a Fab fragment or a single chain Fv fragment.

In certain embodiments, the engineered binding protein comprises an engineered effector function known in the art (see, e.g., Winter, et al. U.S. Pat. Nos. 5,648,260; 5,624,821). The Fc portion of a binding protein mediates several important effector functions e.g. cytokine induction, ADCC, phagocytosis, complement dependent cytotoxicity (CDC) and half-life/clearance rate of binding protein and antigen-binding protein complexes. In some cases these effector functions are desirable for therapeutic binding protein but in other cases might be unnecessary or even deleterious, depending on the therapeutic objectives. Certain human IgG isotypes, particularly IgG1 and IgG3, mediate ADCC and CDC via binding to FcγRs and complement Clq, respectively. Neonatal Fc receptors (FcRn) are the critical components determining the circulating half-life of binding proteins. In still another embodiment at least one amino acid residue is replaced in the constant region of the binding protein, for example the Fc region of the binding protein, such that effector functions of the binding protein are altered.

In certain embodiments, the engineered binding protein is derivatized or linked to another functional molecule (e.g., another peptide or protein). For example, a labeled binding protein of the invention can be derived by functionally linking a binding protein or binding protein portion of the invention (by chemical coupling, genetic fusion, noncovalent association or otherwise) to one or more other molecular entities, such as another binding protein (e.g., a bispecific binding protein or a diabody), a detectable agent, a cytotoxic agent, a pharmaceutical agent, and/or a protein or peptide that can mediate associate of the binding protein with another molecule (such as a streptavidin core region or a polyhistidine tag).

Useful detectable agents with which a binding protein or binding protein portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. A binding protein may also be derivatized with detectable enzymes, such as alkaline phosphatase, horseradish peroxidase, glucose oxidase and the like. When a binding protein is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. A binding protein may also be derivatized with biotin, and detected through indirect measurement of avidin or streptavidin binding.

In other embodiment, the engineered binding protein is further modified to generate glycosylation site mutants in which the O- or N-linked glycosylation site of the binding protein has been mutated. One skilled in the art can generate such mutants using standard well-known technologies. Glycosylation site mutants that retain the biological activity, but have increased or decreased binding activity, are another object of the present invention.

In still another embodiment, the glycosylation of the engineered binding protein or antigen-binding portion of the invention is modified. For example, an aglycoslated binding protein can be made (i.e., the binding protein lacks glycosylation). Glycosylation can be altered to, for example, increase the affinity of the binding protein for antigen. Such carbohydrate modifications can be accomplished by, for example, altering one or more sites of glycosylation within the binding protein sequence. For example, one or more amino acid substitutions can be made that result in elimination of one or more variable region glycosylation sites to thereby eliminate glycosylation at that site. Such aglycosylation may increase the affinity of the binding protein for antigen. Such an approach is described in further detail in PCT Publication WO2003016466A2, and U.S. Pat. Nos. 5,714,350 and 6,350,861, each of which is incorporated herein by reference in its entirety.

Additionally or alternatively, an engineered binding protein of the invention can be further modified with an altered type of glycosylation, such as a hypofucosylated binding protein having reduced amounts of fucosyl residues or a binding protein having increased bisecting GlcNAc structures. Such altered glycosylation patterns have been demonstrated to increase the ADCC ability of binding proteins. Such carbohydrate modifications can be accomplished by, for example, expressing the binding protein in a host cell with altered glycosylation machinery. Cells with altered glycosylation machinery have been described in the art and can be used as host cells in which to express recombinant binding proteins of the invention to thereby produce a binding protein with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342 80, each of which is incorporated herein by reference in its entirety. Using techniques known in the art a practitioner may generate binding proteins exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S. patent Publication Nos. 20040018590 and 20020137134 and PCT publication WO2005100584 A2).

### IV. PRODUCTION OF TNF BINDING PROTEINS

TNF Binding proteins of the present invention may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the binding proteins of the invention in either prokaryotic or eukaryotic host cells, expression of binding proteins in eukaryotic cells is preferable, and most preferable in mammalian host cells, because such eukaryotic cells (and in particular mammalian cells) are more likely than prokaryotic cells to assemble and secrete a properly folded and immunologically active binding protein.

Preferred mammalian host cells for expressing the recombinant binding proteins of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding binding protein genes are introduced into mammalian host cells, the binding proteins are produced by culturing the host cells for a period of time sufficient to allow for expression of the binding protein in the host cells or, more preferably, secretion of the binding protein into the culture medium in which the host cells are grown. Binding proteins can be recovered from the culture medium using standard protein purification methods.

Host cells can also be used to produce functional binding protein fragments, such as Fab fragments or scFv molecules. It will be understood that variations on the above procedure are within the scope of the present invention. For example, it may be desirable to transfect a host cell with DNA encoding functional fragments of either the light chain and/or the heavy chain of a binding protein of this invention. Recombinant DNA technology may also be used to remove some, or all, of the DNA encoding either or both of the light and heavy chains that is not necessary for binding to the antigens of interest. The molecules expressed from such truncated DNA molecules are also encompassed by the binding proteins of the invention. In addition, bifunctional binding proteins may be produced in which one heavy and one light chain are a binding protein of the invention and the other heavy and light chain are specific for an antigen other than the antigens of interest by crosslinking a binding protein of the invention to a second binding protein by standard chemical crosslinking methods.

In a preferred system for recombinant expression of a binding protein, or antigen-binding portion thereof, of the invention, a recombinant expression vector encoding both the binding protein heavy chain and the binding protein light chain is introduced into dhfr-CHO cells by calcium phosphate-mediated transfection. Within the recombinant expression vector, the binding protein heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the binding protein heavy and light chains and intact binding protein is recovered from the culture medium. Standard molecular biology techniques are used to prepare the recombinant expression vector, transfect the host cells, select for transformants, culture the host cells and recover the binding protein from the culture medium. Still further the invention provides a method of synthesizing a recombinant binding protein of the invention by culturing a host cell of the invention in a suitable culture medium until a recombinant binding protein of the invention is synthesized. The method can further comprise isolating the recombinant binding protein from the culture medium.

### V. PHARMACEUTICAL COMPOSITIONS

In one aspect, pharmaceutical compositions comprising one or more binding proteins, either alone or in combination with prophylactic agents, therapeutic agents, and/or pharmaceutically acceptable carriers are provided. The pharmaceutical compositions comprising binding proteins provided herein are for use in, but not limited to, diagnosing, detecting, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, are known to one skilled in the art (US Patent Publication No. 20090311253 A1).

Methods of administering a prophylactic or therapeutic agent provided herein include, but are not limited to, parenteral administration (e.g., intradermal, intramuscular, intraperitoneal, intravenous and subcutaneous), epidural administration, intratumoral administration, mucosal administration (e.g., intranasal and oral routes) and pulmonary administration (e.g., aerosolized compounds administered with an inhaler or nebulizer). The formulation of pharmaceutical compositions for specific routes of administration, and the materials and techniques necessary for the various methods of administration are available and known to one skilled in the art (US Patent Publication No. 20090311253 A1).

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form” refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms provided herein are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a binding protein provided herein is 0.1-20 mg/kg, for example, 1-10 mg/kg. It is to be noted that dosage values may vary with the type and severity of the condition to be alleviated. It is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed composition.

### VI. METHODS OF TREATMENT USING TNF BINDING MOLECULES

In one aspect, provided herein are methods of treating a TNF-associated disorder in a subject by administering to the individual in need of such treatment a therapeutically effective amount a TNF binding molecule disclosed herein. Such methods can be used to treat any TNF-associated disorder including, without limitation:

### A. Sepsis

Tumor necrosis factor has an established role in the pathophysiology of sepsis, with biological effects that include hypotension, myocardial suppression, vascular leakage syndrome, organ necrosis, stimulation of the release of toxic secondary mediators and activation of the clotting cascade (see e.g., Moeller, A., et al. (1990) Cytokine 2:162-169; U.S. Pat. No. 5,231,024 to Moeller et al.; European Patent Publication No. 260 610 B1 by Moeller, A.; Tracey, K. J. and Cerami, A. (1994) Annu. Rev. Med. 45:491-503; Russell, D and Thompson, R. C. (1993) Curr. Opin. Biotech. 4:714-721). Accordingly, a TNF binding protein of the invention can be used to treat sepsis in any of its clinical settings, including septic shock, endotoxic shock, gram negative sepsis and toxic shock syndrome.

Furthermore, to treat sepsis, a combination of the invention can be coadministered with one or more additional therapeutic agents that may further alleviate sepsis, such as an interleukin-1 inhibitor (such as those described in PCT Publication Nos. WO 92/16221 and WO 92/17583), the cytokine interleukin-6 (see e.g., PCT Publication No. WO 93/11793) or an antagonist of platelet activating factor (see e.g., European Patent Application Publication No. EP 374 510). Other combination therapies for the treatment of sepsis are discussed further in herein.

Additionally, in certain embodiments, a TNF binding protein of the invention is administered to a human subject within a subgroup of sepsis patients having a serum or plasma concentration of IL-6 above 500 pg/ml (e.g., above 1000 pg/ml) at the time of treatment (see PCT Publication No. WO 95/20978 by Daum, L., et al.).

### B. Autoimmune Diseases

Tumor necrosis factor has been implicated in playing a role in the pathophysiology of a variety of autoimmune diseases. For example, TNF-alpha has been implicated in activating tissue inflammation and causing joint destruction in rheumatoid arthritis (see e.g., Moeller, A., et al. (1990) Cytokine 2:162-169; U.S. Pat. No. 5,231,024 to Moeller et al.; European Patent Publication No. 260 610 B1 by Moeller, A.; Tracey and Cerami, supra; Arend, W. P. and Dayer, J-M. (1995) Arth. Rheum. 38:151-160; Fava, R. A., et al. (1993) Clin. Exp. Immunol. 94:261-266). TNF-alpha also has been implicated in promoting the death of islet cells and in mediating insulin resistance in diabetes (see e.g., Tracey and Cerami, supra; PCT Publication No. WO 94/08609). TNF-alpha also has been implicated in mediating cytotoxicity to oligodendrocytes and induction of inflammatory plaques in multiple sclerosis (see e.g., Tracey and Cerami, supra). Chimeric and humanized murine anti-hTNF-alpha antibodies have undergone clinical testing for treatment of rheumatoid arthritis (see e.g., Elliott, M. J., et al. (1994) Lancet 344:1125-1127; Elliot, M. J., et al. (1994) Lancet 344:1105-1110; Rankin, E. C., et al. (1995) Br. J. Rheumatol. 34:334-342).

Anti-TNF/JAK inhibitor combinations of the invention can be used to treat autoimmune diseases, in particular those associated with inflammation, including rheumatoid arthritis, rheumatoid spondylitis, osteoarthritis and gouty arthritis, allergy, multiple sclerosis, autoimmune diabetes, autoimmune uveitis and nephrotic syndrome. Typically, the combination is administered systemically, although for certain disorders, local administration of the anti-TNF and/or JAK inhibitor at a site of inflammation may be beneficial (e.g., local administration in the joints in rheumatoid arthritis or topical application to diabetic ulcers, alone or in combination with a cyclohexane-ylidene derivative as described in PCT Publication No. WO 93/19751). Anti-TNF/JAK inhibitor combinations of the invention also can be administered with one or more additional therapeutic agents useful in the treatment of autoimmune diseases, as discussed further herein.

### C. Infectious Diseases

Tumor necrosis factor has been implicated in mediating biological effects observed in a variety of infectious diseases. For example, TNF-alpha has been implicated in mediating brain inflammation and capillary thrombosis and infarction in malaria. TNF-alpha also has been implicated in mediating brain inflammation, inducing breakdown of the blood-brain bather, triggering septic shock syndrome and activating venous infarction in meningitis. TNF-alpha also has been implicated in inducing cachexia, stimulating viral proliferation and mediating central nervous system injury in acquired immune deficiency syndrome (AIDS). Accordingly, the anti-TNF/JAK inhibitor combinations of the invention, can be used in the treatment of infectious diseases, including bacterial meningitis (see e.g., European Patent Application Publication No. EP 585 705), cerebral malaria, AIDS and AIDS-related complex (ARC) (see e.g., European Patent Application Publication No. EP 230 574), as well as cytomegalovirus infection secondary to transplantation (see e.g., Fietze, E., et al. (1994) Transplantation 58:675-680). Anti-TNF/JAK inhibitor combinations of the invention, also can be used to alleviate symptoms associated with infectious diseases, including fever and myalgias due to infection (such as influenza) and cachexia secondary to infection (e.g., secondary to AIDS or ARC).

### D. Transplantation

Tumor necrosis factor has been implicated as a key mediator of allograft rejection and graft versus host disease (GVHD) and in mediating an adverse reaction that has been observed when the rat antibody OKT3, directed against the T cell receptor CD3 complex, is used to inhibit rejection of renal transplants (see e.g., Eason, J. D., et al. (1995) Transplantation 59:300-305; Suthanthiran, M. and Strom, T. B. (1994) New Engl. J. Med. 331:365-375). Accordingly, anti-TNF/JAK inhibitor combinations of the invention, can be used to inhibit transplant rejection, including rejections of allografts and xenografts and to inhibit GVHD. Although the combination may be used alone, it can be used in combination with one or more other agents that inhibit the immune response against the allograft or inhibit GVHD. For example, in one embodiment, a TNF binding protein is used in combination with OKT3 to inhibit OKT3-induced reactions. In another embodiment, a TNF binding protein is used in combination with one or more antibodies directed at other targets involved in regulating immune responses, such as the cell surface molecules CD25 (interleukin-2 receptor-.alpha.), CD11a (LFA-1), CD54 (ICAM-1), CD4, CD45, CD28/CTLA4, CD80 (B7-1) and/or CD86 (B7-2). In yet another embodiment, a TNF binding protein of the invention is used in combination with one or more general immunosuppressive agents, such as cyclosporin A or FK506.

### E. Malignancy

Tumor necrosis factor has been implicated in inducing cachexia, stimulating tumor growth, enhancing metastatic potential and mediating cytotoxicity in malignancies. Accordingly, a TNF binding protein of the invention can be used in the treatment of malignancies, to inhibit tumor growth or metastasis and/or to alleviate cachexia secondary to malignancy. The anti-TNF/JAK inhibitor combination may be administered systemically or locally to the tumor site.

### F. Pulmonary Disorders

Tumor necrosis factor has been implicated in the pathophysiology of adult respiratory distress syndrome (ARDS), including stimulating leukocyte-endothelial activation, directing cytotoxicity to pneumocytes and inducing vascular leakage syndrome. Accordingly, a TNF binding protein of the invention, can be used to treat various pulmonary disorders, including adult respiratory distress syndrome (see e.g., PCT Publication No. WO 91/04054), shock lung, chronic pulmonary inflammatory disease, pulmonary sarcoidosis, pulmonary fibrosis and silicosis. The anti-TNF/JAK inhibitor combination may be administered systemically or locally to the lung surface, for example as an aerosol. An anti-TNF/JAK inhibitor combination of the invention also can be administered with one or more additional therapeutic agents useful in the treatment of pulmonary disorders, as discussed further in herein.

### G. Intestinal Disorders

Tumor necrosis factor has been implicated in the pathophysiology of inflammatory bowel disorders (see e.g., Tracy, K. J., et al. (1986) Science 234:470-474; Sun, X-M., et al. (1988) J. Clin. Invest. 81:1328-1331; MacDonald, T. T., et al. (1990) Clin. Exp. Immunol. 81:301-305). Chimeric murine anti-hTNF-alpha antibodies have undergone clinical testing for treatment of Crohn's disease (van Dullemen, H. M., et al. (1995) Gastroenterology 109:129-135). The anti-TNF/JAK inhibitor combinations of the invention, also can be used to treat intestinal disorders, such as idiopathic inflammatory bowel disease, which includes two syndromes, Crohn's disease and ulcerative colitis. An anti-TNF/JAK inhibitor combination of the invention also can be administered with one or more additional therapeutic agents useful in the treatment of intestinal disorders, as discussed further in herein.

### H. Cardiac Disorders

The anti-TNF/JAK inhibitor combinations of the invention, also can be used to treat various cardiac disorders, including ischemia of the heart (see e.g., European Patent Application Publication No. EP 453 898) and heart insufficiency (weakness of the heart muscle)(see e.g., PCT Publication No. WO 94/20139).

### I. Others Disorders

The anti-TNF/JAK inhibitor combination of the invention, also can be used to treat various other disorders in which TNF-alpha activity is detrimental. Examples of other diseases and disorders in which TNF-alpha activity has been implicated in the pathophysiology, and thus which can be treated using a TNF binding protein of the invention, include inflammatory bone disorders and bone resorption disease (see e.g., Bertolini, D. R., et al. (1986) Nature 319:516-518; Konig, A., et al. (1988) J. Bone Miner. Res. 3:621-627; Lerner, U. H. and Ohlin, A. (1993) J. Bone Miner. Res. 8:147-155; and Shankar, G. and Stern, P. H. (1993) Bone 14:871-876), hepatitis, including alcoholic hepatitis (see e.g., McClain, C. J. and Cohen, D. A. (1989) Hepatology 9:349-351; Felver, M. E., et al. (1990) Alcohol. Clin. Exp. Res. 14:255-259; and Hansen, J., et al. (1994) Hepatology 20:461-474), viral hepatitis (Sheron, N., et al. (1991) J. Hepatol. 12:241-245; and Hussain, M. J., et al. (1994) J. Clin. Pathol. 47:1112-1115), and fulminant hepatitis; coagulation disturbances (see e.g., van der Poll, T., et al. (1990) N. Engl. J. Med. 322:1622-1627; and van der Poll, T., et al. (1991) Prog. Clin. Biol. Res. 367:55-60), burns (see e.g., Giroir, B. P., et al. (1994) Am. J. Physiol. 267:H118-124; and Liu, X. S., et al. (1994) Burns 20:40-44), reperfusion injury (see e.g., Scales, W. E., et al. (1994) Am. J. Physiol. 267:G1122-1127; Serrick, C., et al. (1994) Transplantation 58:1158-1162; and Yao, Y. M., et al. (1995) Resuscitation 29:157-168), keloid formation (see e.g., McCauley, R. L., et al. (1992) J. Clin. Immunol. 12:300-308), scar tissue formation; pyrexia; periodontal disease; obesity and radiation toxicity.

In certain embodiments, an anti-TNF/JAK inhibitor combinations of the invention is used for the treatment of a TNF-associated disorder selected from the group consisting of osteoarthritis, rheumatoid arthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis, scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic polyglandular deficiency type I, polyglandular deficiency type II (Schmidt's syndrome), adult (acute) respiratory distress syndrome, alopecia, alopecia greata, seronegative arthropathy, arthropathy, Reiter's disease, psoriatic arthropathy, ulcerative colitic arthropathy, enteropathic synovitis, Chlamydia-associated arthropathy, Yersinia-associated arthropathy, Salmonella-associated arthropathy, spondyloarthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free disease, chronic mucocutaneous candidiasis, giant cell arteritis, primary sclerosing hepatitis, cryptogenic autoimmune hepatitis, acquired immunodeficiency syndrome, acquired immunodeficiency related diseases, hepatitis B, hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondylitis associated lung disease, vasculitic diffuse lung disease, haemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasculitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjogren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, cholestasis, idiosyncratic liver disease, drug-induced hepatitis, non-alcoholic steatohepatitis, allergy, group B streptococci (GBS) infection, mental disorders (e.g., depression and schizophrenia), Th2 Type and Th1 Type mediated diseases, acute and chronic pain (different forms of pain), cancers such as lung, breast, stomach, bladder, colon, pancreas, ovarian, prostate and rectal cancer and hematopoietic malignancies (leukemia and lymphoma), abetalipoproteinemia, acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, atrial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, antiphospholipid syndrome, anti-receptor hypersensitivity reactions, aortic and peripheral aneurysms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chronic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, dementia pugilistica, demyelinating diseases, dengue hemorrhagic fever, dermatitis, dermatologic conditions, diabetes, diabetic arteriosclerotic disease, diffuse Lewy body disease, dilated congestive cardiomyopathy, disorders of the basal ganglia, Down's syndrome in middle age, drug-induced movement disorders induced by drugs which block CNS dopamine receptors, drug sensitivity, eczema, encephalomyelitis, endocarditis, endocrinopathy, epiglottitis, Epstein-Barr virus infection, erythromelalgia, extrapyramidal and cerebellar disorders, familial hemophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallervorden-Spatz disease, Hashimoto's thyroiditis, hay fever, heart transplant rejection, hemochromatosis, hemodialysis, hemolytic uremic syndrome/thrombolytic thrombocytopenic purpura, hemorrhage, hepatitis A, H is bundle arrhythmias, HIV infection/HIV neuropathy, Hodgkin's disease, hyperkinetic movement disorders, hypersensitivity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, asthenia, infantile spinal muscular atrophy, inflammation of the aorta, influenza A, ionizing radiation exposure, iridocyclitis/uveitis/optic neuritis, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphedema, malaria, malignant lymphoma, malignant histiocytosis, malignant melanoma, meningitis, meningococcemia, metabolic migraine headache, idiopathic migraine headache, mitochondrial multisystem disorder, mixed connective tissue disease, monoclonal gammopathy, multiple myeloma, multiple systems degenerations (Menzel, Dejerine-Thomas, Shy-Drager, and Machado-Joseph), myasthenia gravis, mycobacterium avium intracellulare, mycobacterium tuberculosis, myelodysplastic syndrome, myocardial infarction, myocardial ischemic disorders, nasopharyngeal carcinoma, neonatal chronic lung disease, nephritis, nephrosis, neurodegenerative diseases, neurogenic muscular atrophies, neutropenic fever, non-Hodgkin's lymphoma, occlusion of the abdominal aorta and its branches, occlusive arterial disorders, orchitis/epididymitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherosclerotic disease, peripheral vascular disorders, peritonitis, pernicious anemia, pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, progressive supranucleo palsy, primary pulmonary hypertension, radiation therapy, Raynaud's phenomenon, Raynaud's disease, Refsum's disease, regular narrow QRS tachycardia, renovascular hypertension, reperfusion injury, restrictive cardiomyopathy, sarcomas, senile chorea, senile dementia of Lewy body type, seronegative arthropathies, shock, sickle cell anemia, skin allograft rejection, skin changes syndrome, small bowel transplant rejection, solid tumors, specific arrhythmias, spinal ataxia, spinocerebellar degenerations, streptococcal myositis, structural lesions of the cerebellum, subacute sclerosing panencephalitis, syncope, syphilis of the cardiovascular system, systemic anaphylaxis, systemic inflammatory response syndrome, systemic onset juvenile rheumatoid arthritis, telangiectasia, thromboangiitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, viral encephalitis/aseptic meningitis, viral-associated hemophagocytic syndrome, Wernicke-Korsakoff syndrome, Wilson's disease, xenograft rejection of any organ or tissue, acute coronary syndromes, acute idiopathic polyneuritis, acute inflammatory demyelinating polyradiculoneuropathy, acute ischemia, adult Still's disease, alopecia greata, anaphylaxis, anti-phospholipid antibody syndrome, aplastic anemia, arteriosclerosis, atopic eczema, atopic dermatitis, autoimmune dermatitis, autoimmune disorder associated with streptococcus infection, autoimmune enteropathy, autoimmune hearing loss, autoimmune lymphoproliferative syndrome (ALPS), autoimmune myocarditis, autoimmune premature ovarian failure, blepharitis, bronchiectasis, bullous pemphigoid, cardiovascular disease, catastrophic antiphospholipid syndrome, celiac disease, cervical spondylosis, chronic ischemia, cicatricial pemphigoid, clinically isolated syndrome (CIS) with risk for multiple sclerosis, childhood onset psychiatric disorder, chronic obstructive pulmonary disease (COPD), dacryocystitis, dermatomyositis, diabetic retinopathy, disk herniation, disk prolapse, drug induced immune hemolytic anemia, endocarditis, endometriosis, endophthalmitis, episcleritis, erythema multiforme, erythema multiforme major, gestational pemphigoid, Guillain-Barre syndrome (GBS), hay fever, Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratojunctivitis sicca, Kussmaul disease or Kussmaul-Meier disease, Landry's paralysis, Langerhan's cell histiocytosis, livedo reticularis, macular degeneration, microscopic polyangiitis, Morbus Bechterev, motor neuron disorders, mucous membrane pemphigoid, multiple organ failure, myasthenia gravis, myelodysplastic syndrome, myocarditis, nerve root disorders, neuropathy, non-A non-B hepatitis, optic neuritis, osteolysis, ovarian cancer, pauciarticular JRA, peripheral artery occlusive disease (PAOD), peripheral vascular disease (PVD), peripheral artery disease (PAD), phlebitis, polyarteritis nodosa (or periarteritis nodosa), polychondritis, polymyalgia rheumatica, poliosis, polyarticular JRA, polyendocrine deficiency syndrome, polymyositis, polymyalgia rheumatica (PMR), post-pump syndrome, primary Parkinsonism, prostate and rectal cancer and hematopoietic malignancies (leukemia and lymphoma), prostatitis, pure red cell aplasia, primary adrenal insufficiency, recurrent neuromyelitis optica, restenosis, rheumatic heart disease, SAPHO (synovitis, acne, pustulosis, hyperostosis, and osteitis), secondary amyloidosis, shock lung, scleritis, sciatica, secondary adrenal insufficiency, silicone associated connective tissue disease, Sneddon-Wilkinson dermatosis, spondylitis ankylosans, Stevens-Johnson syndrome (SJS), systemic inflammatory response syndrome, temporal arteritis, toxoplasmic retinitis, toxic epidermal necrolysis, transverse myelitis, TRAPS (tumor-necrosis factor receptor type 1 (TNFR)-associated periodic syndrome), type 1 allergic reaction, type II diabetes, urticaria, usual interstitial pneumonia (UIP), vasculitis, vernal conjunctivitis, viral retinitis, Vogt-Koyanagi-Harada syndrome (VKH syndrome), and wet macular degeneration. In a particular embodiment, the TNF-associated disease or disorder is rheumatoid arthritis.

### VII. DIAGNOSTICS

The disclosure herein also provides diagnostic applications including, but not limited to, diagnostic assay methods, diagnostic kits containing one or more TNF binding proteins, and adaptation of the methods and kits for use in automated and/or semi-automated systems. The methods, kits, and adaptations provided may be employed in the detection, monitoring, and/or treatment of a disease or disorder in an individual. This is further elucidated below.

### Method of Assay

The present disclosure also provides a method for determining the presence, amount or concentration of an analyte, or fragment thereof, in a test sample using at least one binding protein as described herein. Any suitable assay as is known in the art can be used in the method. Examples include, but are not limited to, immunoassays and/or methods employing mass spectrometry.

Immunoassays provided by the present disclosure may include sandwich immunoassays, radioimmunoassay (RIA), enzyme immunoassay (EIA), enzyme-linked immunosorbent assay (ELISA), competitive-inhibition immunoassays, fluorescence polarization immunoassay (FPIA), enzyme multiplied immunoassay technique (EMIT), bioluminescence resonance energy transfer (BRET), and homogenous chemiluminescent assays, among others.

A chemiluminescent microparticle immunoassay, in particular one employing the ARCHITECT® automated analyzer (Abbott Laboratories, Abbott Park, Ill.), is an example of an immunoassay.

Methods employing mass spectrometry are provided by the present disclosure and include, but are not limited to MALDI (matrix-assisted laser desorption/ionization) or by SELDI (surface-enhanced laser desorption/ionization).

Methods for collecting, handling, processing, and analyzing biological test samples using immunoassays and mass spectrometry would be well-known to one skilled in the art, are provided for in the practice of the present disclosure (US 2009-0311253 A1).

### Kit

A kit for assaying a test sample for the presence, amount or concentration of an analyte, or fragment thereof, in a test sample is also provided. The kit comprises at least one component for assaying the test sample for the analyte, or fragment thereof, and instructions for assaying the test sample for the analyte, or fragment thereof. The at least one component for assaying the test sample for the analyte, or fragment thereof, can include a composition comprising a binding protein, as disclosed herein, and/or an anti-analyte binding protein (or a fragment, a variant, or a fragment of a variant thereof), which is optionally immobilized on a solid phase.

Optionally, the kit may comprise a calibrator or control, which may comprise isolated or purified analyte. The kit can comprise at least one component for assaying the test sample for an analyte by immunoassay and/or mass spectrometry. The kit components, including the analyte, binding protein, and/or anti-analyte binding protein, or fragments thereof, may be optionally labeled using any art-known detectable label. The materials and methods for the creation provided for in the practice of the present disclosure would be known to one skilled in the art (US 2009-0311253 A1).

### Adaptation of Kit and Method

The kit (or components thereof), as well as the method of determining the presence, amount or concentration of an analyte in a test sample by an assay, such as an immunoassay as described herein, can be adapted for use in a variety of automated and semi-automated systems (including those wherein the solid phase comprises a microparticle), as described, for example, in U.S. Pat. Nos. 5,089,424 and 5,006,309, and as commercially marketed, for example, by Abbott Laboratories (Abbott Park, Ill.) as ARCHITECT®.

Other platforms available from Abbott Laboratories include, but are not limited to, AxSYM®, IMx® (see, for example, U.S. Pat. No. 5,294,404, PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. Nos. 5,063,081, 7,419,821, and 7,682,833; and US Publication Nos. 20040018577, 20060160164 and US 20090311253

### VIII. EXEMPLIFICATION

The following examples are included for purposes of illustration only and are not intended to be limiting of the invention.

### Example 1

### Internalization of Bivalent TNF Binding Molecules by Monocytic Cells

### Isolation of Monocytes, Culture and Stimulation:

Peripheral blood mononuclear cells (PBMC) were isolated from leukopack of healthy donors by density gradient centrifugation over Ficoll-Paque (GE Health Sciences). Monocytes were isolated by magnetic sorting using CD14 microbeads (Mitenyi Biotec). The purity of the resulting monocytes, as assessed by flow cytometric analysis, was typically greater than 98%. Monocytes were cultured in RPMI1640 medium (Cellgro) supplemented with 2 mM L-glutamine, 100 μg/ml penicillin, and streptomycin, and 10% fetal bovine serum at a density of 1×10⁶ cells/ml at 37° C. with 5% CO2. To test the surface TNFalpha expression, PBMCs or monocytes were stimulated with ultra-low (0.025 ng/ml), low (0.25 ng/ml) or high (250 ng/ml) of LPS (from Salmonella typhimurium, Sigma-Aldrich) for indicated period.

### Dendritic Cell Differentiation and Stimulation

Dendritic cells were generated by culturing monocytes in RPMI1640 medium supplemented with 100 ng/ml of recombinant human GM-CSF (Abbvie) and 5 ng/ml of human IL-4 (Peprotech) for 4 days. To investigate the TNFalpha production DCs were stimulated with 1 ug/ml of LPS (from Salmonella typhimurium, Sigma-Aldrich) for indicated period.

### Staining Cells and Flow Cytometric Analysis

LPS stimulated PBCs, Monocyte or DCs were stained with Anti-TNFalpha specific monoclonal antibodies AB436 (MAK195-AM21), AB437 (MAK195-AM24), AB441 (MAK199-AM1) or AB444 (MAK199-AM4) for 1 hour on ice. As a negative control an isotype matched control antibody (AB446) was used. All the antibodies were conjugated with A488 using antibody labeling kit (Invitrogen) according to manufacturer's protocol. Monocytes and T cells were gated based on the expression of CD14 (Biolegend) and CD3 (eBioscience) respectively. Samples were analyzed on a Becton Dickinson Fortessa flow cytometer, and analysis was performed using Flowjo software (TreeStar Inc., Ashland, Oreg., USA).

### Internalization Assay

To investigate the internalization of surface TNF bound antibodies, monocytes were stimulated with LPS for 4, 7, 9 or 24 hours in the presence of Alexa 488 conjugated AB436 antibodies. Cells were permeabilized and nucleus was stained with DAPI. The images were acquired using confocal microscope (Zeiss). To study the internalization of anti-TNF antibodies by dendritic cells, the monocyte derived DCs were stimulated with LPS for 4 hours in the presence of anti-TNF (AB441) or matched isotype control antibodies. The Anti-TNFalpha specific antibodies and control antibodies were conjugated with pH sensitive dye pHRodo Red (Invitrogen) according to manufacturer's protocol. The cells were analyzed by fluorescent microscope and FACS. Where indicated, the surface of the cells was stained with A488-conjugated anti-HLA-A,B.C (W6/32, Biolegend) antibodies and the nucleus was stained with Nuce blue (Invitrogen). To study the internalization kinetics of anti-TNFalpha antibodies by membrane TNF on DCs, cells were either left in un-stimulated or stimulated with LPS for 1 hour or 24 hours. The surface TNFalpha was stained with pHRodo Red conjugated anti-TNFalpha antibody (AB441). The stained cells were cultured in RPMI medium for indicated time and the internalization was assessed as increase in fluorescence using BD Fortessa flow cytometer.

### Cell Surface Biotinylation

Cells (2−3×10⁶) were washed twice with ice-cold PBS-CM (PBS containing 1 mM CaCl₂ and 1 mM MgCl₂) and the cell surface proteins derivatized twice by using 1 mg/ml cell-impermeable EZ-Link-Sulfo-NHS-SS-Biotin in PBS-CM on ice for 30 min protected from light with gentle agitation. Excess biotin was quenched by incubating the cells for 10 min on ice in 50 mM NH₄Cl. Cells were washed twice with PBS-CM and total proteins extracted in 150 l lysis buffer (50 mM Tris-HCl, pH 7.4, 150 mM NaCl, 50 mM n-Octyl-β-D-glucoside, 0.5% sodium deoxycholate, 1 tablet EDTA-free protease inhibitor cocktail in 7 ml lysis buffer, 1 mM PMSF) on ice for 45 min and centrifuged at 12,000×g for 10 min at 4° C. Clarified supernatant was transferred to a fresh microcentrifuge tube on ice and total proteins estimated by the BCA (Bicinchoninic acid) protein assay reagent. To enrich the cell surface biotinylated proteins, 25-75 ug total proteins were transferred to fresh tube and the volume made to 500 ul using the lysis buffer and mixed with 50 ul streptavidin-conjugated agarose beads. The tube was mixed end-over-end at 4° C. overnight. The agarose beads were collected by centrifugation at 2,500×g for 3 min and sequentially washed twice by suspending in 1 ml fresh ice-cold lysis buffer, once in 1 ml ice-cold 500 mM NaCl, and once in 1 ml 50 mM Tris-HCl, pH 8. The streptavidin-agarose bound, cell surface biotinylated proteins, along with 6-15 ug total proteins in a separate tube, were suspended in 40 ul SDS-PAGE sample buffer containing 4M urea and 5% b-mercaptoethanol, separated on 4-20% Novex Tris-Glycine SDS-PAGE, and transferred onto a 0.2 um nitrocellulose membrane for 1 h. The nitrocellulose membrane was incubated in 5% non-fat dry milk in TBS-T (25 mM Tris-HCl, 150 mM NaCl, pH 7.5, containing 0.2% Tween-20) for 30 min at room temperature with gentle agitation, washed once in TBS-T for 5 min at room temperature and incubated overnight with gentle agitation at 4° C. in the following primary antibodies: (1) Rabbit-Pan Cadherin IgG (1:1000 in 5% bovine serum albumin, BSA, in TBS-T); (2) FITC Mouse anti-Human CD14 IgG (1:500 in 5% non-fat dry milk, in TBS-T); (3) Human anti-Human TNF-a, hMAK199 AM4, IgG (1:1000 in 5% non-fat dry milk, in TBS-T); and (4) Rabbit anti-GAPDH IgG (1:5000 in 5% non-fat dry milk in TBS-T)

The next day, the membrane was washed twice for 15 min each with TBS-T with vigorous agitation at room temperature. The membrane was incubated in the appropriate horseradish peroxidase (HRP)-conjugated secondary IgG in 5% non-fat dry milk in TBS-T for 45 at room temperature with gentle agitation and washed twice for 15 min each in TBS-T with vigorous agitation at room temperature. The membrane was incubated either in ECL or ECL Prime western blotting analysis systems and exposed to X-ray films for various periods of time.

### Reagents Used in Cell Surface Biotinylation

### EZ-Link Sulfo-NHS-SS-Biotin (Thermo Scientific Pierce, USA; Catalog #21331)

### Streptavidin Agarose Resin (Thermo Scientific Pierce, USA; Catalog #20347)

### Triton X-100 (Sigma Aldrich, USA; Catalog #T-9284)

n-Octyl-β-D-glucoside (Thermo Scientific Pierce, USA; Catalog #28310)
EDTA-free protease inhibitor cocktail (Roche Diagnostics, USA; Catalog #11836170001)
Sodium deoxycholate (Sigma Aldrich, USA; Catalog #D6750)

### BCA Protein Assay Reagent (Thermo Scientific Pierce, USA; Catalog #23225)

Streptavidin agarose beads (Thermo Scientific Pierce, USA; Catalog #20347)
PMSF, Phenylmethanesulfonyl fluoride (Sigma Aldrich, USA; Catalog #78830)

### Novex 4-20% Gel (Life Technologies, USA; Catalog #EC6028BOX)

0.2 um Nitrocellulose membrane (Life Technologies, USA; Catalog #LC2000)

### Tween 20 (Sigma Aldrich, USA; Catalog #P9416)

FITC Mouse anti-Human CD14 IgG (BD Pharmingen, USA; Catalog #555397)

### Bovine Serum Albumin, BSA (Thermo Scientific Pierce, USA; Catalog #37525)

### Rabbit Pan-Cadherin IgG (Cell Signaling Technology, USA; Catalog #4068)

Rabbit anti-GAPDH IgG (Cell Signaling Technology, USA; Catalog #2118)
Human anti-Human TNF hMAK199 AM4 IgG (Abbvie)
Anti-Human IgG HRP linked whole antibody from Sheep (GE Healthcare, UK; Catalog #NA933V)

### ECL Western Blotting Analysis System (GE Healthcare, UK; Catalog #RPN2108)

### ECL Western Blotting Detection Reagent (GE Healthcare, UK; Catalog #RPN2232)

### Amersham Hyperfilm ECL (GE Healthcare, UK; Catalog #28906836)

### Construction of Monovalent Molecules

The dual variable domain immunoglobulin (DVD-Ig) molecule is designed such that two different light chain variable domains (VL) from the two different parent monoclonal antibodies are linked in tandem directly or via a short linker by recombinant DNA techniques, followed by the light chain constant domain and, optionally, an Fc region. Similarly, the heavy chain comprises two different heavy chain variable domains (VH) linked in tandem, followed by the constant domain CH1 and Fc region. The poly-Ig molecule is designed in one instance to incorporate a swapped CH1 with CL constant region, or a VH plus CH with VL plus CL.

The variable domains is obtained using recombinant DNA techniques from a parent antibody generated by any one of the methods described herein. In certain embodiments, the variable domain is a CDR grafted or a humanized variable heavy or light chain domain. In certain embodiments, the variable domain is a human heavy or light chain variable domain.

In certain embodiments the first and second variable domains are linked directly to each other using recombinant DNA techniques. In certain embodiments the variable domains are linked via a linker sequence. In certain embodiments, two variable domains are linked together. The variable domains may bind the same antigen or may bind different antigens. Poly-Ig molecules of the invention may include one immunoglobulin variable domain and one non-immunoglobulin variable domain such as ligand binding domain of a receptor, active domain of an enzyme. Poly-Ig molecules may also comprise 2 or more non-Ig domains.

The linker sequence can be a single amino acid or a polypeptide sequence. In certain embodiment, the linker sequences are selected from the group consisting of AKTTPKLEEGEFSEAR (SEQ ID NO:); AKTTPKLEEGEFSEARV (SEQ ID NO:); AKTTPKLGG (SEQ ID NO:); SAKTTPKLGG (SEQ ID NO:); SAKTTP (SEQ ID NO:); RADAAP (SEQ ID NO:); RADAAPTVS (SEQ ID NO:); RADAAAAGGPGS (SEQ ID NO:); RADAAAA(G₄S)₄ (SEQ ID NO:); SAKTTPKLEEGEFSEARV (SEQ ID NO:); ADAAP (SEQ ID NO:); ADAAPTVSIFPP (SEQ ID NO:); TVAAP (SEQ ID NO:); TVAAPSVFIFPP (SEQ ID NO:); QPKAAP (SEQ ID NO:); QPKAAPSVTLFPP (SEQ ID NO:); AKTTPP (SEQ ID NO:); AKTTPPSVTPLAP (SEQ ID NO:); AKTTAP (SEQ ID NO:); AKTTAPSVYPLAP (SEQ ID NO:); ASTKGP (SEQ ID NO:); ASTKGPSVFPLAP (SEQ ID NO:), GGGGSGGGGSGGGGS (SEQ ID NO:); GENKVEYAPALMALS (SEQ ID NO:); GPAKELTPLKEAKVS (SEQ ID NO:); GHEAAAVMQVQYPAS (SEQ ID NO:), TVAAPSVFIFPPTVAAPSVFIFPP (SEQ ID NO:); and ASTKGPSVFPLAPASTKGPSVFPLAP (SEQ ID NO:). In addition poly-Igs that swap the inner domain utilize a hybridized long or short linker that combines a heavy and light chain transition for the heavy chain and a light chain to heavy chain transition for the light chain and consist of ASTKGPSVFIFPP (SEQ IN NO. X); ASTVAP (SEQ ID NO. X); TVAAPSVFPLAP (SED ID NO. X); and TVASTP 9SEQ ID NO. X).

The choice of linker sequences is based on crystal structure analysis of several Fab molecules. There is a natural flexible linkage between the variable domain and the CH1/CL constant domain in Fab or antibody molecular structure. This natural linkage comprises approximately 10-12 amino acid residues, contributed by 4-6 residues from C-terminus of V domain and 4-6 residues from the N-terminus of CL/CH1 domain. DVD Igs of the invention were generated using N-terminal 5-6 amino acid residues, or 11-12 amino acid residues, of CL or CH1 as linker in light chain and heavy chain of DVD-Ig, respectively. The N-terminal residues of CL or CH1 domains, particularly the first 5-6 amino acid residues, adopt a loop conformation without strong secondary structures; therefore can act as flexible linkers between the two variable domains. The N-terminal residues of CL or CH1 domains are natural extension of the variable domains, as they are part of the Ig sequences, therefore minimize to a large extent any immunogenicity potentially arising from the linkers and junctions.

Other linker sequences may include any sequence of any length of CL/CH1 domain but not all residues of CL/CH1 domain; for example the first 5-12 amino acid residues of the CL/CH1 domains; the light chain linkers can be from Cκ or Cλ; and the heavy chain linkers can be derived from CH1 of any isotypes, including Cγ1, Cγ2, Cγ3, Cγ4, Cα1, Cα2, Cδ, Cε, and Cμ. Linker sequences may also be derived from other proteins such as Ig-like proteins, (e.g., TCR, FcR, KIR); G/S based sequences (e.g., G4S repeats SEQ ID NO: 29); hinge region-derived sequences; and other natural sequences from other proteins.

In certain embodiments, a constant domain is linked to the two linked variable domains using recombinant DNA techniques. In an embodiment, sequence comprising linked heavy chain variable domains is linked to a heavy chain constant domain and sequence comprising linked light chain variable domains is linked to a light chain constant domain. In an embodiment, the constant domains are human heavy chain constant domain and human light chain constant domain respectively. In an embodiment, the DVD heavy chain is further linked to an Fc region. The Fc region may be a native sequence Fc region, or a variant Fc region. In another embodiment, the Fc region is a human Fc region. In another embodiment the Fc region includes Fc region from IgG1, IgG2, IgG3, IgG4, IgA, IgM, IgE, or IgD.

### Transfection and Expression in 293 Cells

Expression of the reference molecules was accomplished by transiently co-transfecting HEK293 (EBNA) cells with plasmids containing the corresponding light-chains (LC) and heavy-chains (HC) nucleic acids. HEK293 (EBNA) cells were propagated in Freestyle 293 media (Invitrogen, Carlsbad Calif.) at a 0.5 L-scale in flasks (2L Corning Cat #431198) shaking in a CO2 incubator (8% CO2, 125 RPM, 37° C.). When the cultures reached a density of 1×106 cells/ml, cells were transfected with transfection complex. Transfection complex was prepared by first mixing 150 μg LC-plasmids and 100 μg HC-plasmids together in 25 ml of Freestyle media, followed by the addition of 500 ul PEI stock solution [stock solution: 1 mg/ml (pH 7.0) Linear 25 kDa PEI, Polysciences Cat #23966]. The transfection complex was mixed by inversion and allowed to incubate at room temperature for 20 minutes prior to being added to the cell culture. Following transfection, cultures continued to be grown in the CO₂ incubator (8% CO₂, 125 RPM, 37° C.). Twenty-four hours after transfection, the culture was supplemented with 25 ml of a 10% Tryptone N1 solution (Organo Technie, La Courneuve France Cat #19553). Nine days after transfection, cells were removed from the cultures by centrifugation (16,000 g, 10 minutes), and the retained supernatant was sterile filtered (Millipore HV Durapore Stericup, 0.45 um) and placed at 4° C. until initiation of the purification step.

Each molecule was individually purified using a disposable 2 ml packed column (packed by Orochem Technologies) containing MabSelect SuRe resin (GE Healthcare). Columns were pre-equilibriated in PBS and then loaded with the harvested 0.55 L samples overnight (15 hours) at 1 ml/minute with the flow-through being recirculated back into the feed container. Following the loading step, columns were washed with 20 ml PBS and protein was eluted by feeding elution buffer [50 mM Citric acid pH 3.5] at 4 ml/min and collecting fractions (1 ml) in tubes already containing 0.2 ml of 1.5M Tris pH 8.2 (bringing the final pH to approximately 6.0). Fractions containing antibody were pooled based on the chromatograms and dialyzed into the final storage buffer [10 mM citric acid, 10 mM Na₂HPO₄, pH 6.0]. Following dialysis, samples were filtered through a 0.22 um Steriflip (Millipore) and the protein concentration was determined by absorbance [Hewlett Packard 8453 diode array spectrophotometer]. SDS-PAGE analysis was performed on analytical samples (both reduced and non-reduced) to assess final purity, verify the presence of appropriately sized heavy- and light-chain bands, and confirm the absence of significant amounts of free (e.g., uncomplexed) light chain (in the non-reduced samples) and mis-paired poly-Igs.

### Size Exclusion Chromatography

Molecules are diluted to 2.5 ug/mL with water and 20 mL is analyzed on a Shimadzu HPLC system using a TSK gel G3000 SWXL column (Tosoh Bioscience, cat #k5539-05k). Samples are eluted from the column with 211 mM sodium sulfate, 92 mM sodium phosphate, pH 7.0, at a flow rate of 0.3 mL/minutes. The HPLC system operating conditions are the following:

Mobile phase: 211 mM Na₂SO₄, 92 mM Na₂HPO₄.7H₂O, pH 7.0

### Gradient: Isocratic

Flow rate: 0.3 mL/minute
Detector wavelength: 280 nm
Autosampler cooler temp: 4° C.
Column oven temperature: Ambient
Run time: 50 minutes

### SDS-PAGE

Molecules are analyzed by sodium dodecyl sulfate—polyacrylamide gel electrophoresis (SDS-PAGE) under both reducing and denaturing conditions. For reducing conditions, the samples are mixed 1:1 with 2× tris glycine SDS-PAGE sample buffer (Invitrogen, cat #LC2676, lot #1323208) with 100 mM DTT, and heated at 90° C. for 10 minutes in the presence of BME (beta-mercaptoethanol). For denaturing conditions, the samples are mixed 1:1 with sample buffer and heated at 90° C. for 10 minutes. The reduced and denatured samples (10 g per lane) are loaded on a 12% pre-cast tris-glycine gel (Invitrogen, cat #EC6005box, lot #6111021). See Blue Plus 2 (Invitrogen, cat #LC5925, lot #1351542) is used as a molecular weight marker. The gels are run in a XCell SureLock mini cell gel box (Invitrogen, cat #EI0001) and the proteins are separated by first applying a voltage of 75 to stack the samples in the gel, followed by a constant voltage of 125 until the dye front reached the bottom of the gel. The running buffer used is 1× tris glycine SDS buffer, prepared from a 10× tris glycine SDS buffer (ABC, MPS-79-080106)). The gels are stained overnight with colloidal blue stain (Invitrogen cat #46-7015, 46-7016) and destained with Milli-Q water until the background is clear. The stained gels are then scanned using an Epson Expression scanner (model 1680, S/N DASX003641).

### Affinity Determination Using BIACORE Technology

The BIACORE assay (Biacore, Inc, Piscataway, N.J.) determines the affinity of antibodies or Poly-Ig with kinetic measurements of on-rate and off-rate constants. Binding of antibodies or Poly-Ig to a target antigen (for example, a purified recombinant target antigen) is determined by surface plasmon resonance-based measurements with a BiacoreR 1000 or 3000 instrument (Biacore® AB, Uppsala, Sweden) using running HBS-EP (10 mM HEPES [pH 7.4], 150 mM NaCl, 3 mM EDTA, and 0.005% surfactant P20) at 25° C. All chemicals are obtained from Biacore® AB (Uppsala, Sweden) or otherwise from a different source as described in the text. For example, approximately 5000 RU of goat anti-mouse IgG, (Fcγ), fragment specific polyclonal antibody (Pierce Biotechnology Inc, Rockford, Ill.) diluted in 10 mM sodium acetate (pH 4.5) is directly immobilized across a CMS research grade biosensor chip using a standard amine coupling kit according to manufacturer's instructions and procedures at 25 μg/ml. Unreacted moieties on the biosensor surface are blocked with ethanolamine. Modified carboxymethyl dextran surface in flowcell 2 and 4 is used as a reaction surface. Unmodified carboxymethyl dextran without goat anti-mouse IgG in flow cell 1 and 3 is used as the reference surface. For kinetic analysis, rate equations derived from the 1:1 Langmuir binding model are fitted simultaneously to association and dissociation phases of all eight injections (using global fit analysis) with the use of Biaevaluation 4.0.1 software. Purified antibodies or Poly-Ig are diluted in HEPES-buffered saline for capture across goat anti-mouse IgG specific reaction surfaces. Antibodies or Poly-Ig to be captured as a ligand (25 μg/ml) are injected over reaction matrices at a flow rate of 5 μl/min. The association and dissociation rate constants, kₒₙ (M⁻¹ s⁻¹) and k_{off} (s⁻¹) are determined under a continuous flow rate of 25 μl/min. Rate constants are derived by making kinetic binding measurements at different antigen concentrations ranging from 10⁻200 nM. The equilibrium dissociation constant (M) of the reaction between antibodies or Poly-Igs and the target antigen is then calculated from the kinetic rate constants by the following formula: K_{D}=k_{off}/kₒₙ. Binding is recorded as a function of time and kinetic rate constants are calculated. In this assay, on-rates as fast as 10⁶ M⁻¹ s⁻¹ and off-rates as slow as 10⁻⁶ s⁻¹ can be measured.

### Results

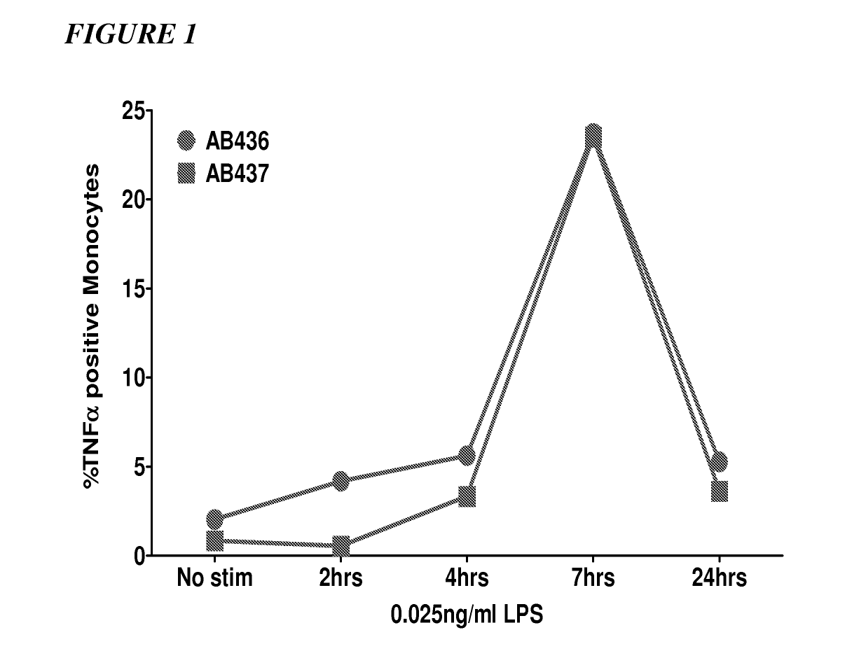

Peripheral blood mononuclear cells were stimulated with 0.025 ng/ml of LPS for indictade time period. The TNFalpha present on the surface were stained with anti-TNFAalpha antibody (AB436 and AB437). The frequency of TNFalpha positive cells were plotted against the time of incubation. The monocytes were gated based on CD14 expression. These results are set forth in FIG. 1.

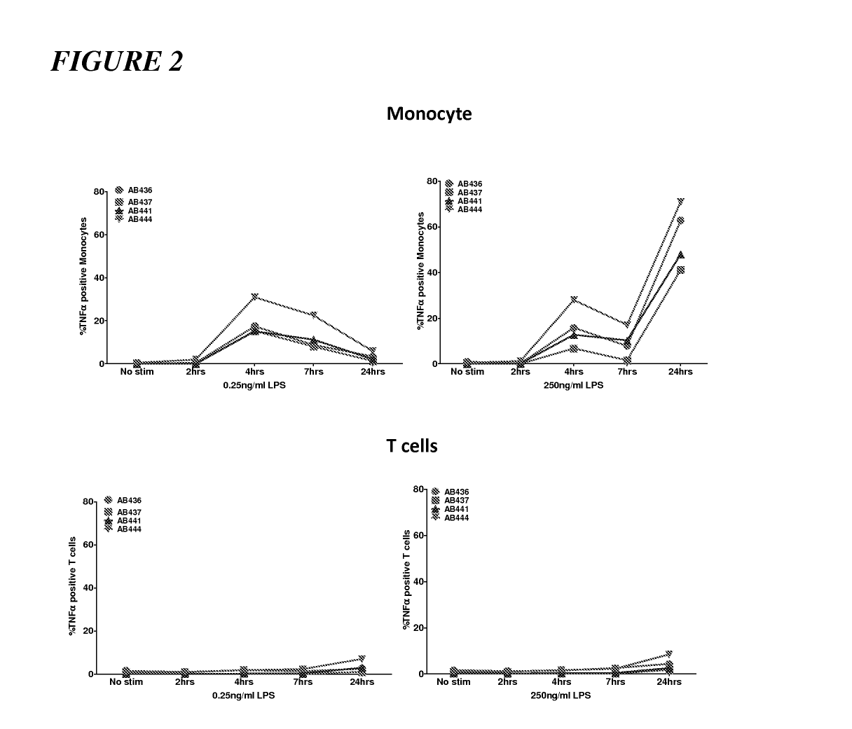

Peripheral blood mononuclear cells were stimulated with 0.25 ng/ml or 250 ng/ml of LPS for indictade time period. The TNFalpha present on the surface was stained with anti-TNFalphaA antibody (AB436, AB437, AB441 and AB444). The frequency of TNFalpha positive cells were plotted against the time of incubation. The monocytes were gated based on CD14 expression and the T cells were gated on CD3 expression. These results are set forth in FIG. 2.

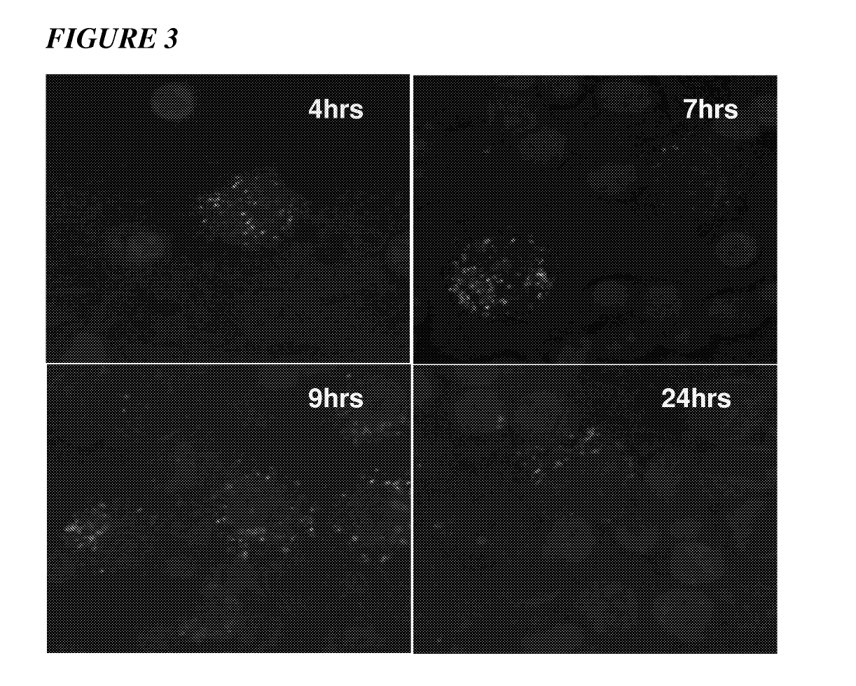

Peripheral blood mononuclear cells were stimulated with 0.25 ng/ml LPS for indictade time period in the presence of Alexa488 conjugated Anti-TNFalpha (AB436) antibody (green). The cells were permeabilized and nucleau was stained with DAPI (blue). These results are set forth in FIG. 3.

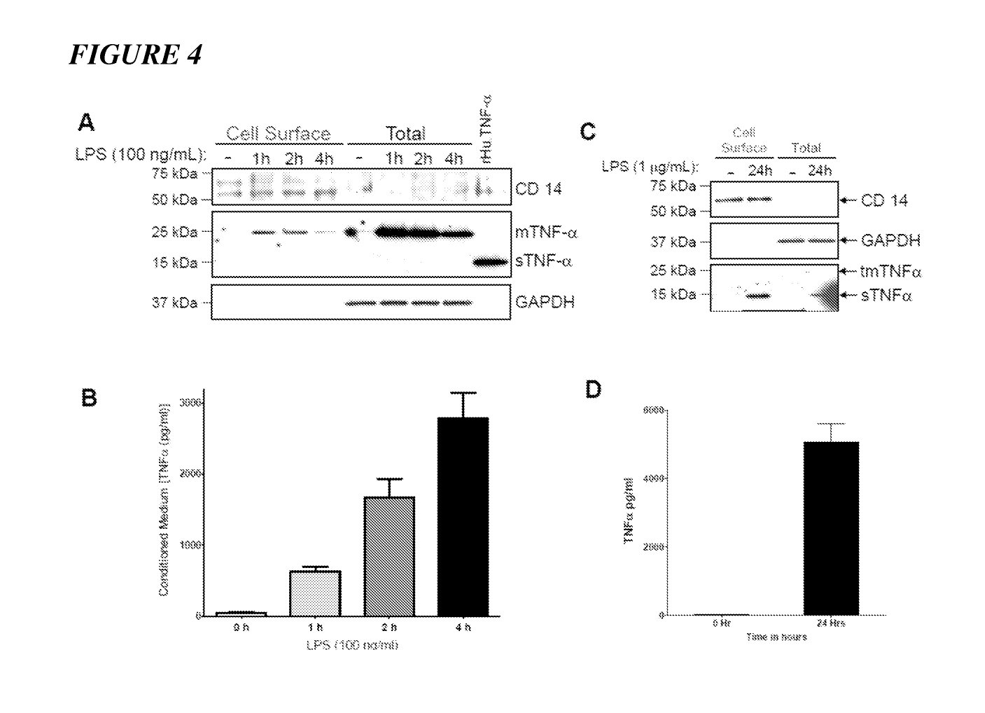

FIG. 4 shows the TNFalpha expression in LPS treated human monocytes. A. Cell Surface-associated TNF-a: CD14+ human monocytes were either untreated or treated with 100 ng/mL LPS for the indicated period of time. Cell surface proteins were derivatized using cell-impermeable Sulfo-NHS-SS-Biotin, total proteins extracted in detergent-containing buffer, and cell surface biotinylated proteins enriched on streptavidin-agarose. Surface-biotinylated and total proteins were resolved on SDS-PAGE and subjected to immunoblotting using anti-human TNFalpha IgG. CD14 and GAPDH expressions were used as cell surface and cytoplasmic protein loading controls, respectively. tm=Transmembrane, s=Soluble. B. Levels of Soluble TNFalpha: Conditioned media from CD14+ human monocytes treated as in (A) were assayed for soluble TNFalpha. (C) Surface expression TNFalpha was assessed as in (A) after 24 hours stimulation of monocytes with LPS. (D) The superatants from (C) were analyzed for soluble TNFalpha

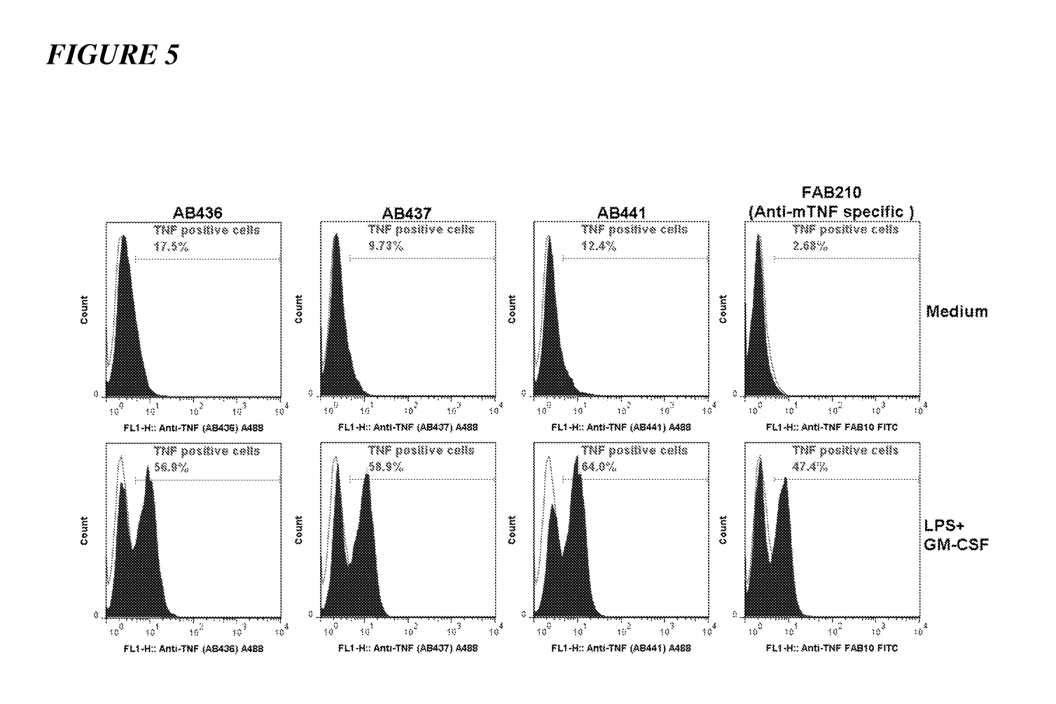

FIG. 5 shows surface TNFalpha expression on peripheral blood monocytes stimulated with GM-CSF and LPS. Peripheral blood monocytes were stimulated with 1 ug/ml of LPS and 100 ng/ml of recombinant human GM-CSF for 24 hours. The TNFalpha present on the surface was stained with anti-TNFalpha antibody (Filled histogram: AB436, AB437, AB441 and FAB210) or matched isotype control antibody (red open histogram).

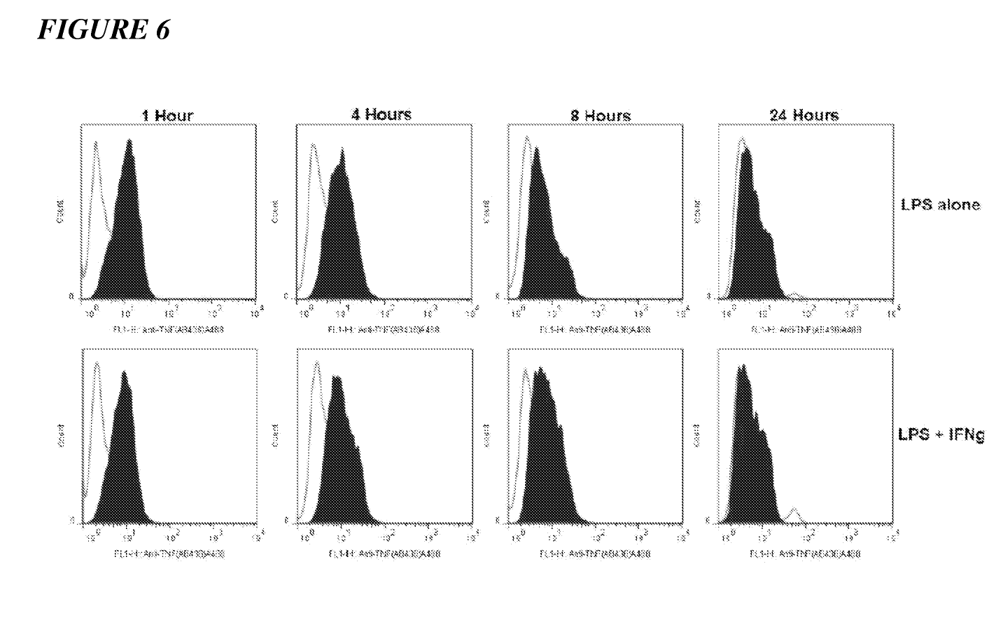

Peripheral blood monocytes were cultured in medium supplemented with recombinant human GM-CSF (100 ng/ml) and 5 ng/ml IL-4 for 4 days. The cells were stimulated with 1 ug/ml of LPS in the presence or absence of 10 ng/ml IFNalpha for indicated time period. The TNFalpha present on the surface was stained with anti-TNFalpha antibody (Filled histogram: AB436) or matched isotype control antibody (red open histogram). These results are set forth in FIG. 6.

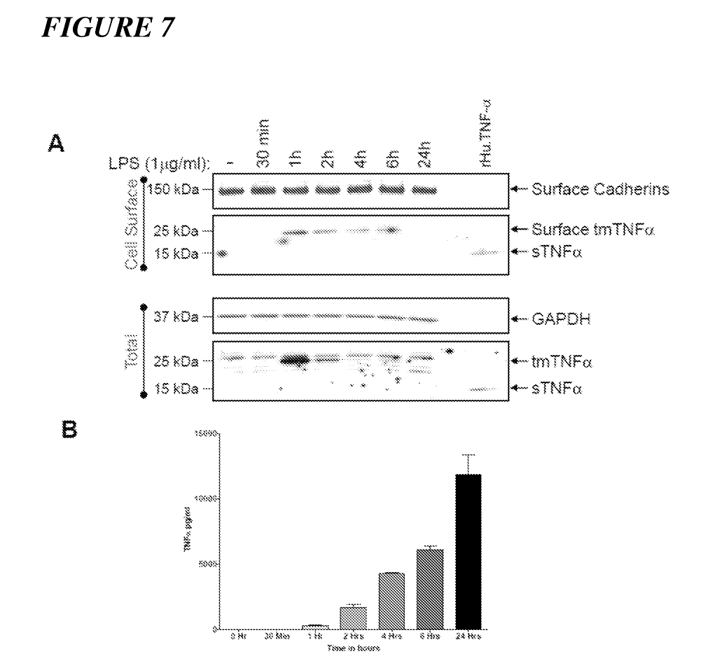

FIG. 7 shows TNFalpha expression in LPS treated human monocyte derived dendritic cells. A. Cell Surface-associated TNFalphaA: Human dendritic cells were either left untreated or treated with 1 ug/mL LPS for the indicated period of time. Cell surface proteins were derivatized using cell-impermeable Sulfo-NHS-SS-Biotin, total proteins extracted in detergent-containing buffer, and cell surface biotinylated proteins enriched on streptavidin-agarose. Surface-biotinylated and total proteins were resolved on SDS-PAGE and subjected to immunoblotting using anti-human TNFalpha IgG. Cadherins and GAPDH expressions were used as cell surface and cytoplasmic protein loading controls, respectively. tm=Transmembrane, s=Soluble. B. Levels of Soluble TNFalpha: Conditioned media from human dendritic cells treated as in (A) were assayed for soluble TNFalpha.

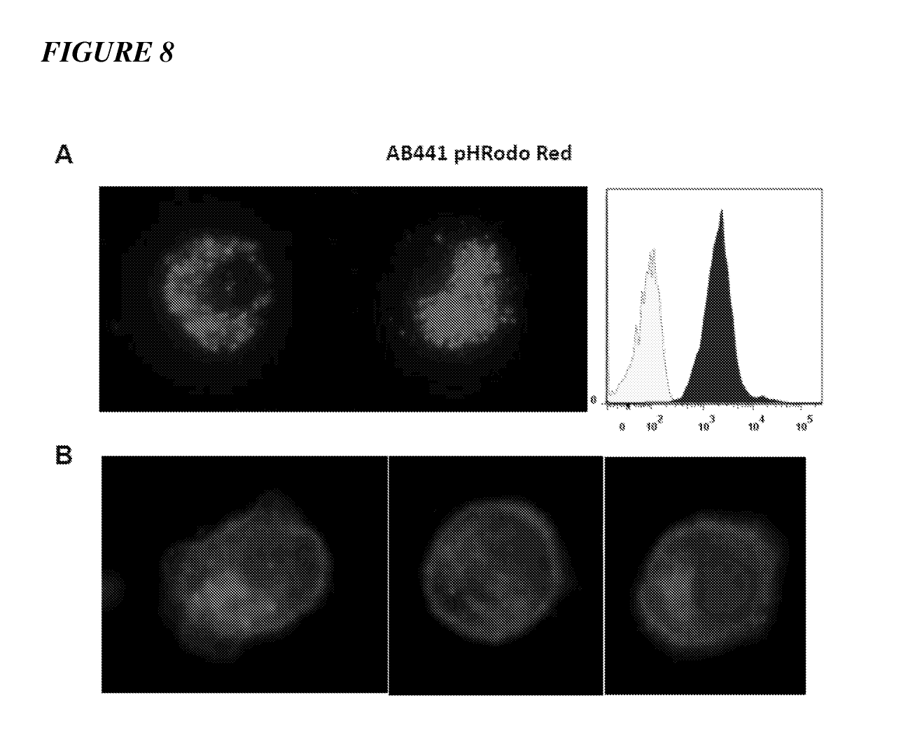

Peripheral blood monocytes were cultured in medium supplemented with recombinant human GM-CSF (100 ng/ml) and 5 ng/ml IL-4 for 4 days. (A) The cells were stimulated with 1 ug/ml of LPS in the presence Anti-TNFalpha (AB441) antibodies conjugated with pHRodo Red dye (blue filled histogram, red punctate staining in microscopy) for 4 hours or matched isotype control antibody conjugated with the same dye (red dotted histogram). (B) The cells were treated as in (A) for 4 hours with last 20 minutes with nuce blue stain to stain the nuleus (blue). The cells were washed and stained with MHC Class I on the surface (Green) to mark the surface. The internalized anti-TNFalpha antibody (red: AB441) were visualized by fluorescent microscopy. These results are set forth in FIG. 8.

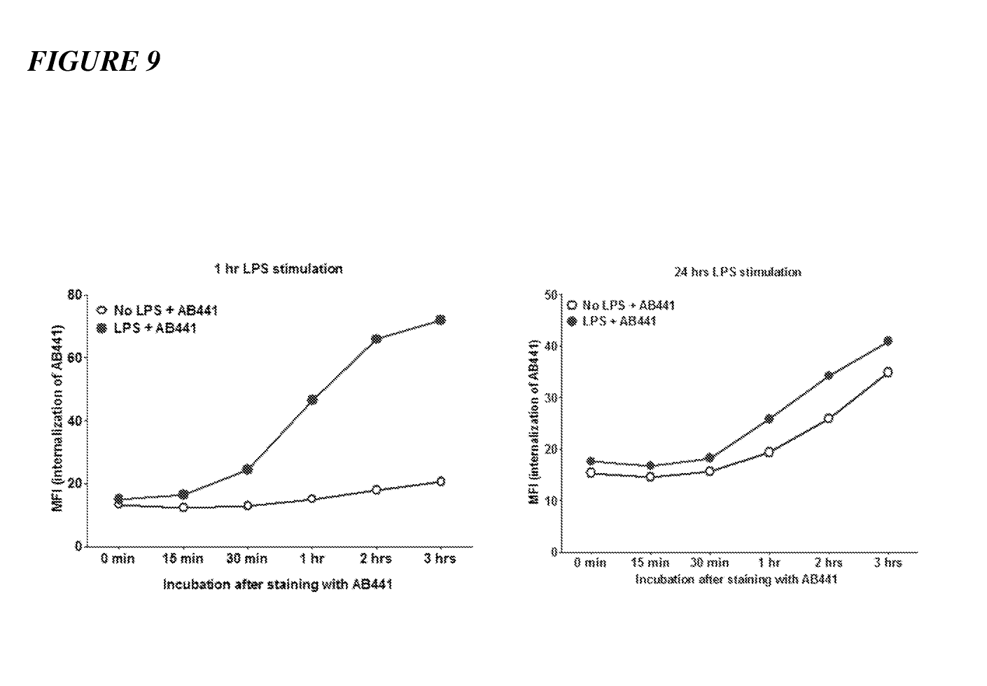

Peripheral blood monocytes were cultured in medium supplemented with recombinant human GM-CSF (100 ng/ml) and 5 ng/ml IL-4 for 4 days. The cells were left unstimulated (blue open circle) or stimulated (red filled circle) with 1 ug/ml of LPS for 1 hour (Left panel) or 24 hours (right panel). The cells were harvested and stained wifor surface TNFa with anti-TNFalpha (AB441) antibodies conjugated with pHRodo Red dye. T cells were cultured in medium for indicated time and the internalization was measures as increase in fluorescent intensity using BD Fortessa flow cytometer. These results are set forth in FIG. 9.

Taken together these data demonstrate that bivalent TNF binding proteins are internalized by monocytic cells.

### Example 2

### Exemplary Monovalent Antibody Formats

### Half-Bodies

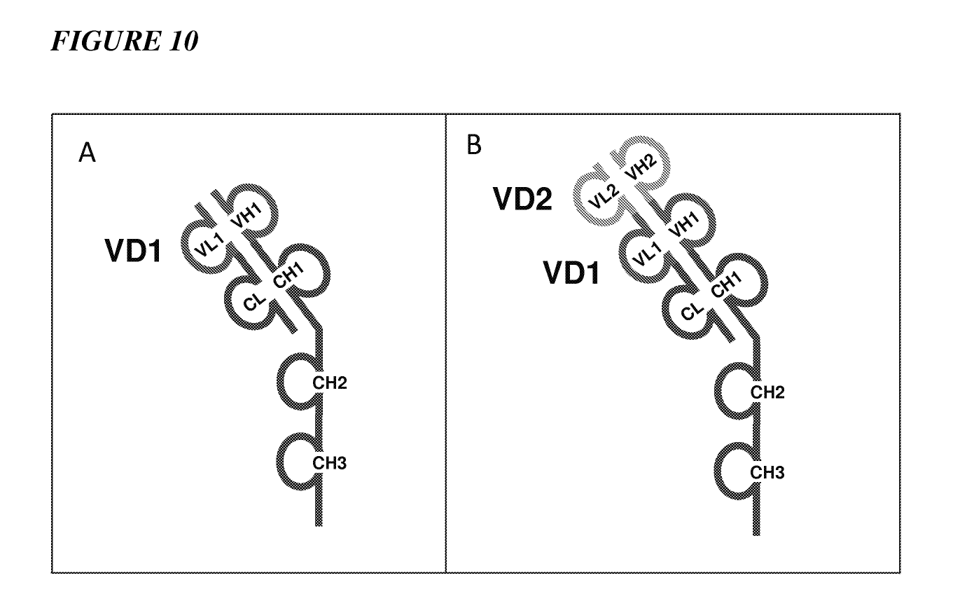

Half body molecules describe a monoclonal antibody (A) or dual-variable-domain immunoglobulin (B) containing an Fc region with C227S, C230S, F405R mutations (according to the EU numbering convention). These mutations prevent the formation of an antibody tetramer by inhibiting the disulfide bonding between heavy chains. The resulting molecules are comprised of one heavy and one light chain dimer of a mAb or DVD-Ig capable of monovalent binding to the variable domain's antigen. In the case of the DVD-Ig half body, the molecule may be designed to contain two distinct variable domains, or two variable domains binding the same target. Half body mAbs may be comprised of any VH and VL pair for anti-TNF. Half body DVD-Ig may be comprised of any combination of VH/VL variable domain pairs between anti-TNF and anti-IL-17 (Table: “Examples of half body anti-TNF molecules”), or others, with linker combinations of long-long, long-short, short-short, and GS10 (see linker table). Exemplary half-body molecules are depicted in FIG. 10.

### Abbmab

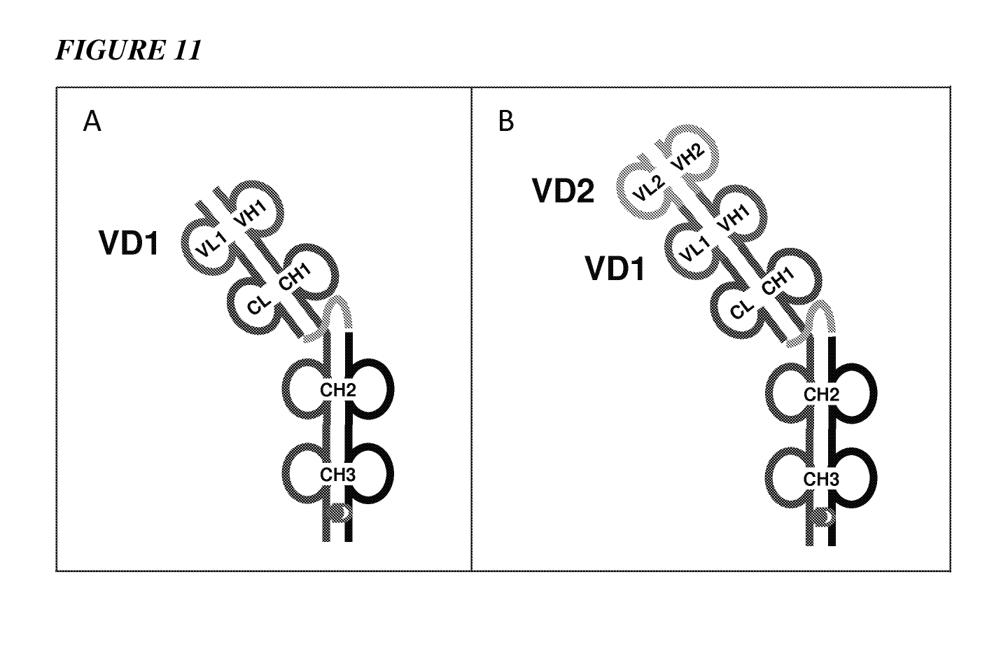

Abbvie-mAbs (Abbmab) molecules describe a monoclonal antibody (A) or dual-variable-domain immunoglobulin (B) containing an Fc with CH3 hole mutations (See Table 3). In addition to the CH3-hole on one heavy chain, the light chain contains a linker sequence attached to a CH2 and CH3 with knob mutation. This molecule dimerizes to form one heavy chain paired to one light chain and one CH2-CH3 chain. This allows for the formation of an intact Fc linked to a monovalent binding domain. Abbmabs may be comprised of any VH and VL pair for anti-TNF. Abbmav DVD-Igs may be comprised of any combination of VH/VL variable domain pairs between e.g., anti-TNF and other variable domains (Exemplary anti-TNF variable domains, linkers are set forth in Table 2 and 1, respectively). Exemplary abbmab molecules are depicted in FIG. 11.

### M-Bodies

Monovalent immunoglobulin (m-body) molecules describe a monoclonal antibody (A) or dual-variable-domain immunoglobulin (B) containing an Fc with CH3 knob-into-holes mutations (See Table 3). However, monovalency is achieved by the mutations of key residues within the CDRs of the heavy chain. This allows for the sharing of the light chain between two similar heavy chains with one chain not active against the antigen of interest.

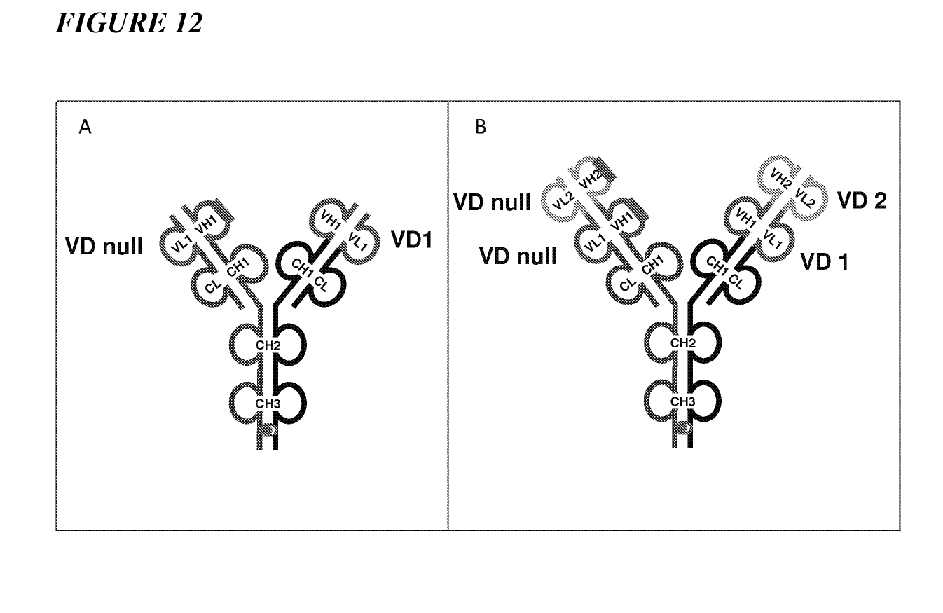

M-bodies may be comprised of any VH and VL pair for anti-TNF. M-body DVD-Igs may be comprised of any combination of VH/VL variable domain (Exemplary anti-TNF variable domains, linkers are set forth in Table 2 and 1, respectively). In addition DVD-Igs may also contain two variable domains active against antigen on the same arm with knock out mutations for both on the CDRs of the opposite arm. In addition, M-body DVDs may also contain a bivalent domain paired with a monovalent domain (designed by CDR mutation) where the monovalent domain is anti-TNF. Exemplary m-body molecules are depicted in FIG. 12.

### Poly-Ig Molecules

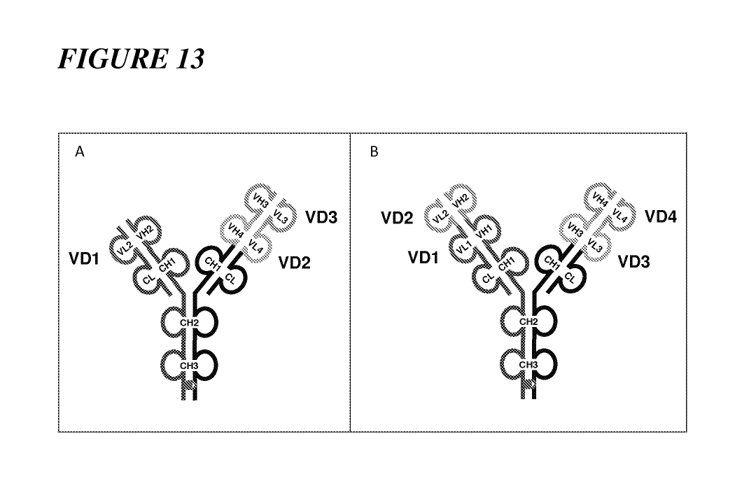

Multivariable, monovalent anti-TNF poly-Ig molecules may combine 3 independent variable domains or 2 bivalent domains combined with one monovalent domain (A). In addition a multivariable, monovalent molecule may combine 4 independent variable domains or 1 bivalent and 2 monovalent domains (B). Each format contains an Fc with CH3 knob-into-holes mutations (See Table 3). In addition bivalent and monovalent domains may be positioned between heavy chain Fabs or within them, with possibly different outcomes for each orientation. Exemplary molecules are depicted in FIG. 13

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

We claim:

1. A binding protein that specifically binds to human TNF, wherein the binding protein comprises an antibody variable region and an Fc region, and wherein and the binding protein exhibits an amount of cellular internalization upon binding to cell surface human TNF that is less than the amount of cellular internalization exhibited by an anti-human TNF reference antibody.

2. The binding protein of claim 1, wherein the reference antibody is infliximab, adalimumab, or golimumab.

3. The binding protein of claim 1, which binds monovalently to cell surface human TNF on antigen presenting cells.

4. The binding protein of claim 1, comprising a first polypeptide chain and a second polypeptide chain, wherein the first polypeptide chain comprises VDH-(X1)n-C—Y1, wherein
VDH is a heavy chain variable domain,
X1 is a linker with the proviso that it is not CH1,
C is a CH1 domain,
Y1 is an Fc region,
n is 0 or 1;
and wherein the second polypeptide chains comprises VDL-(X3)m-C, wherein
VDL is a light chain variable domain,
X3 is a linker with the proviso that it is not CH1,
C is a CL1,
m is 0 or 1;
wherein X2 comprises at least one mutation that inhibits dimerization of Y1.

5. The binding protein of claim 4, wherein Y1 comprises an amino acid sequence selected from the group set forth Table 3.

6. The binding protein of claim 4, wherein X1 and/or X3 comprises an amino acid sequence set forth in Table 1.

7. The binding protein of claim 4, wherein VDH comprises the heavy chain CDRs or complete VH domain amino acid sequence of infliximab, adalimumab, certolizumab pegol, or golimumab.

8. The binding protein of claim 4, wherein VDL comprises the light chain CDRs or complete VL domain amino acid sequence of infliximab, adalimumab, certolizumab pegol, or golimumab.

9. The binding protein of claim 1, comprising a first polypeptide chain and a second polypeptide chain, wherein the first polypeptide chain comprises VDH1-(X1)n-VDH2-X2-(X3)m-Y1, wherein:
VDH1 is a first heavy chain variable domain;
X1 is a linker with the proviso that X1 is not CH1;
VDH2 is a second heavy chain variable domain;
X2 is CH1;
X3 is a linker;
Y1 is an Fc region;
n is 0 or 1, m is 0 or 1;
and wherein the second polypeptide chain comprises VDL1-(X4)m-VDL2-X5, wherein:
VDL1 is a first light chain variable domain;
X4 is a linker with the proviso that X4 is not CH1;
VDL2 is a second light chain variable domain;
X5 is CL1;
m is 0 or 1, wherein Y1 comprises at least one mutation that inhibits homodimerization of Y1.

10. The binding protein of claim 9, wherein X1, X2, and/or X4 comprises an amino acid sequence set forth in Table 1.

11. The binding protein of claim 9, wherein Y1 comprises an amino acid sequence set forth in Table 3.

12. The binding protein of claim 9, wherein VDH1 and/or VDH2 comprises the heavy chain CDRs or complete VH domain amino acid sequence of infliximab, adalimumab, certolizumab pegol, or golimumab.

13. The binding protein of claim 9, wherein VDL1 and/or VDL2 comprises the light chain CDRs or complete VL domain amino acid sequence of infliximab, adalimumab, certolizumab pegol, or golimumab.

14. The binding protein of claim 1, comprising four polypeptide chains, wherein two of said four polypeptide chains comprise VDH-(X1)n-C—Y1, wherein VDH is a heavy chain variable domain,
X1 is a linker with the proviso that it is not CH1,
C is a CH1 domain,
Y1 is an Fc region,
n is 0 or 1;
and wherein two of said four polypeptide chains comprise VDL-(X2)m—X3, wherein
VDL is a light chain variable domain,
X2 is a linker with the proviso that it is not CH1,
X3 is a CL domain,
m is 0 or 1; wherein at least one of said four polypeptide chains comprises a mutation, said mutation being located in the variable domain, wherein said mutation inhibits the targeted binding between the specific antigen and the mutant binding domain.

15. The binding protein of claim 14, wherein Y1 comprises a mutation that enhances heterodimerization.

16. The binding protein of claim 14, wherein Y1 comprises an amino acid sequence set forth in Table 3.

17. The binding protein of claim 14, wherein X1 and/or X2 comprises an amino acid sequence set forth Table 1.

18. The binding protein of claim 14, wherein VDH comprises the heavy chain CDRs or complete VH domain amino acid sequence of infliximab, adalimumab, certolizumab pegol, or golimumab.

19. The binding protein of claim 14, wherein VDL comprises the light chain CDRs or complete VL domain amino acid sequence of infliximab, adalimumab, certolizumab pegol, or golimumab.

20. The binding protein of claim 1, comprising four polypeptide chains, wherein two of said four polypeptide chains comprise VDH1-(X1)n-VDH2-C—Y1, wherein
VDH1 is a first heavy chain variable domain,
VDH2 is a second heavy chain variable domain,
C is a heavy chain constant domain,
X1 is a linker with the proviso that it is not CH1,
Y1 is an Fc region,
n is 0 or 1;
and wherein two of said four polypeptide chains comprise VDL1-(X2)m-VDL2-X3, wherein
VDL1 is a first light chain variable domain,
VDL2 is a second light chain variable domain,
X2 is a linker with the proviso that it is not CH1,
X3 is a CL domain,
m is 0 or 1, wherein at least one of said four polypeptide chains comprises a mutation, said mutation being located in the first variable domain or the second variable domain, wherein said mutation inhibits the targeted binding between the specific antigen and the mutant binding domain.

21. The binding protein of claim 20, wherein the mutation is located in VDH1 and/or VDH2.

22. The binding protein of claim 20, wherein the mutation is located in VDL1 and/or VDL2.

23. The binding protein of claim 20, wherein Y1 comprises a mutation that enhances heterodimerization.

24. The binding protein of claim 20, wherein Y1 comprises an amino acid sequence set forth in Table 3.

25. The binding protein of claim 20, wherein X1 and/or X2 comprises and amino acid sequence set forth in Table 1.

26. The binding protein of claim 20, wherein VDH1 and/or VDH2 comprises the heavy chain CDRs or complete VH domain amino acid sequence of infliximab, adalimumab, certolizumab pegol, or golimumab.

27. The binding protein of claim 20, wherein VDL1 and/or VDL2 comprises the light chain CDRs or complete VL domain amino acid sequence of infliximab, adalimumab, certolizumab pegol, or golimumab.

28. The binding protein of claim 1, comprising a first polypeptide chain and a second polypeptide chain, said first polypeptide chain comprising VDH-(X1)n-X2-(X3)m-Y1, wherein:
VDH is a heavy chain variable domain;
X1 is a linker with the proviso that X1 is not CH1;
X2 is CH1;
X3 is a linker;
Y1 is an F region;
n is 0 or 1, m is 0 or 1;
and said second polypeptide comprising VDL-(X4)n-X5-(X6)m-Y2, wherein:
VDL is a light chain variable domain;
X4 is a linker with the proviso that X4 is not CH1;
X5 is CL1;
X6 is a linker;
Y2 is an F region;
n is 0 or 1, m is 0 or 1, wherein Y1 and Y2 each comprises a mutation, wherein the mutations on Y1 and Y2 enhance the interaction between Y1 and Y2.

29. The binding protein of claim 28, wherein Y1 and/or Y2 comprises an amino acid sequence set forth in Table 3.

30. The binding protein of claim 28, wherein X1, X3, X4, and/or X6 comprises and amino acid sequence set forth in Table 1.

31. The binding protein of claim 28, wherein VDH comprises the heavy chain CDRs or complete VH domain amino acid sequence of infliximab, adalimumab, certolizumab pegol, or golimumab.

32. The binding protein of claim 28, wherein VDL comprises the light chain CDRs or complete VL domain amino acid sequence of infliximab, adalimumab, certolizumab pegol, or golimumab.

33. The binding protein of claim 1, comprising a first polypeptide chain and a second polypeptide chain, said first polypeptide chain comprising VDH1-(X1)n-VDH2-X2-(X3)m-Y1, wherein:
VDH1 is a first heavy chain variable domain;
X1 is a linker with the proviso that X1 is not CH1;
VDH2 is a second heavy chain variable domain;
X2 is CH1;
X3 is a linker;
Y1 is an F region;
n is 0 or 1, m is 0 or 1;
and said second polypeptide comprising VDL1-(X4)n-VDL2-X5-(X6)m-Y2, wherein:
VDL1 is a first light chain variable domain;
X4 is a linker with the proviso that X4 is not CH1;
VDL2 is a second light chain variable domain;
X5 is CL1;
X6 is a linker;
Y2 is an F region;
n is 0 or 1, m is 0 or 1, wherein Y1 and Y2 each comprises a mutation, wherein the mutations on Y1 and Y2 enhance heterodimerization between Y1 and Y2.

34. The binding protein of claim 33, wherein Y1 and/or Y2 comprises an amino acid sequence set forth in Table 3.

35. The binding protein of claim 33, wherein X1 and/or X3, comprises and amino acid sequence set forth in Table 1.

36. The binding protein of claim 33, wherein VDH1 and/or VDH2 comprises the heavy chain CDRs or complete VH domain amino acid sequence of infliximab, adalimumab, certolizumab pegol, or golimumab.

37. The binding protein of claim 33, wherein VDL1 and/or VDL2 comprises the light chain CDRs or complete VL domain amino acid sequence of infliximab, adalimumab, certolizumab pegol, or golimumab.

38. The binding protein of claim 1, comprising first, second, third and fourth polypeptide chains,
wherein said first polypeptide chain comprises VD1-(X1)n-VD2-CH—(X2)n, wherein VD1 is a first heavy chain variable domain, VD2 is a second heavy chain variable domain, C is a CH1 domain, X1 is a linker with the proviso that it is not a constant domain, and X2 is an Fc region;
wherein said second polypeptide chain comprises VD1-(X1)n-VD2-CL-(X2)n, wherein VD1 is a first light chain variable domain, VD2 is a second light chain variable domain, CL is a light chain constant domain, X1 is a linker with the proviso that it is not a constant domain, and X2 does not comprise an Fc region;
wherein said third polypeptide chain comprises VD3-(X3)n-VD4-CL-(X4)n, wherein VD3 is a third heavy chain variable domain, VD4 is a fourth heavy chain variable domain, CL is a light chain constant domain, X3 is a linker with the proviso that it is not a constant domain, and X4 is an Fc region;
wherein said fourth polypeptide chain comprises VD3-(X3)n-VD4-CH—(X4)n, wherein VD3 is a third light chain variable domain, VD4 is a fourth light chain variable domain, CH is CH1 domain, X3 is a linker with the proviso that it is not a constant domain, and X4 does not comprise an Fc region;
wherein n is 0 or 1, and wherein the VD1 domains on the first and second polypeptide chains form one functional binding site for a first antigen, the VD2 domains on the first and second polypeptide chains form one functional binding site for a second antigen, the VD3 domains on the third and fourth polypeptide chains form one functional binding site for a third antigen, and the VD4 domains on the third and fourth polypeptide chains form one functional binding site for forth antigen.

39. The binding protein of claim 38, wherein at least one of the first, second, third or forth antigens is human TNF.

40. The binding protein of claim 38, wherein X2 and/or X4 comprises at least one mutation that enhances heterodimerization of X2 and X4.

41. The binding protein of claim 38, wherein X2 and/or X4 comprises an amino acid sequence set forth in Table 3.

42. The binding protein of claim 38, wherein X1 and/or X3, comprises and amino acid sequence set forth in Table 1.

43. The binding protein of claim 38, wherein VD1, VD2, VD3, and/or VD4 comprise the heavy chain CDRs, the light chain CDRs, the complete VH domain, or the complete VL domain amino acid sequence of infliximab, adalimumab, certolizumab pegol, or golimumab.

44. The binding protein of claim 1, comprising a polypeptide chain, wherein the polypeptide chain comprises RD1-(X)n-VDH-C—Y or VDH-(X)n-RD1-C—Y, wherein
RD1 comprises a ligand-binding domain of a receptor;
VDH is a heavy chain variable domain;
C is a heavy chain constant domain;
X is a linker with the proviso that it is not CH1;
Y is an Fc region; and
n is 0 or 1.

45. The binding protein of claim 44, wherein RD1 comprises a receptor that binds to human TNF.

46. The binding protein of claim 44, wherein RD1 comprises the TNF receptor binding portion of etanercept.

47. The binding protein of claim 44, wherein VDH comprises the heavy chain CDRs, or the complete VH domain acid sequence of infliximab, adalimumab, certolizumab pegol, or golimumab.

48. A composition comprising a binding polypeptide of claim 1 and a pharmaceutically acceptable carrier or excipient.

49. A method of treating a TNF-associated disorder in a subject in need thereof, comprising administering to the subject an effective amount of the composition of claim 48.

50. An isolated polynucleotide encoding the binding polypeptide of claim 1.

51. A vector comprising the polynucleotide of claim 50.

52. A host cell comprising the vector of claim 51.


SEQUENCE LISTING (60 sequences)

SEQ ID NO: 1 - protein, 6 aa, Artificial Sequence
  Description of Artificial Sequence Synthetic peptide
ASTKGP

SEQ ID NO: 2 - protein, 5 aa, Artificial Sequence
  Description of Artificial Sequence Synthetic peptide
TVAAP

SEQ ID NO: 3 - protein, 12 aa, Artificial Sequence
  Description of Artificial Sequence Synthetic peptide
TVAAPSVFIFPP

SEQ ID NO: 4 - protein, 13 aa, Artificial Sequence
  Description of Artificial Sequence Synthetic peptide
ASTKGPSVFPLAP

SEQ ID NO: 5 - protein, 6 aa, Artificial Sequence
  Description of Artificial Sequence Synthetic peptide
GGGGSG

SEQ ID NO: 6 - protein, 5 aa, Artificial Sequence
  Description of Artificial Sequence Synthetic peptide
GGSGG

SEQ ID NO: 7 - protein, 13 aa, Artificial Sequence
  Description of Artificial Sequence Synthetic peptide
QEPKSSDKTHTSP

SEQ ID NO: 8 - protein, 16 aa, Artificial Sequence
  Description of Artificial Sequence Synthetic peptide
AKTTPKLEEGEFSEAR

SEQ ID NO: 9 - protein, 17 aa, Artificial Sequence
  Description of Artificial Sequence Synthetic peptide
AKTTPKLEEGEFSEARV

SEQ ID NO: 10 - protein, 9 aa, Artificial Sequence
  Description of Artificial Sequence Synthetic peptide
AKTTPKLGG

SEQ ID NO: 11 - protein, 10 aa, Artificial Sequence
  Description of Artificial Sequence Synthetic peptide
SAKTTPKLGG

SEQ ID NO: 12 - protein, 6 aa, Artificial Sequence
  Description of Artificial Sequence Synthetic peptide
SAKTTP

SEQ ID NO: 13 - protein, 6 aa, Artificial Sequence
  Description of Artificial Sequence Synthetic peptide
RADAAP

SEQ ID NO: 14 - protein, 9 aa, Artificial Sequence
  Description of Artificial Sequence Synthetic peptide
RADAAPTVS

SEQ ID NO: 15 - protein, 12 aa, Artificial Sequence
  Description of Artificial Sequence Synthetic peptide
RADAAAAGGPGS

SEQ ID NO: 16 - protein, 27 aa, Artificial Sequence
  Description of Artificial Sequence Synthetic peptide
RADAAAAGGGGSGGGGSGGGGSGGGGS

SEQ ID NO: 17 - protein, 18 aa, Artificial Sequence
  Description of Artificial Sequence Synthetic peptide
SAKTTPKLEEGEFSEARV

SEQ ID NO: 18 - protein, 5 aa, Artificial Sequence
  Description of Artificial Sequence Synthetic peptide
ADAAP

SEQ ID NO: 19 - protein, 12 aa, Artificial Sequence
  Description of Artificial Sequence Synthetic peptide
ADAAPTVSIFPP

SEQ ID NO: 20 - protein, 5 aa, Artificial Sequence
  Description of Artificial Sequence Synthetic peptide
TVAAP

SEQ ID NO: 21 - protein, 12 aa, Artificial Sequence
  Description of Artificial Sequence Synthetic peptide
TVAAPSVFIFPP

SEQ ID NO: 22 - protein, 6 aa, Artificial Sequence
  Description of Artificial Sequence Synthetic peptide
QPKAAP

SEQ ID NO: 23 - protein, 13 aa, Artificial Sequence
  Description of Artificial Sequence Synthetic peptide
QPKAAPSVTLFPP

SEQ ID NO: 24 - protein, 6 aa, Artificial Sequence
  Description of Artificial Sequence Synthetic peptide
AKTTPP

SEQ ID NO: 25 - protein, 13 aa, Artificial Sequence
  Description of Artificial Sequence Synthetic peptide
AKTTPPSVTPLAP

SEQ ID NO: 26 - protein, 6 aa, Artificial Sequence
  Description of Artificial Sequence Synthetic peptide
AKTTAP

SEQ ID NO: 27 - protein, 13 aa, Artificial Sequence
  Description of Artificial Sequence Synthetic peptide
AKTTAPSVYPLAP

SEQ ID NO: 28 - protein, 6 aa, Artificial Sequence
  Description of Artificial Sequence Synthetic peptide
ASTKGP

SEQ ID NO: 29 - protein, 13 aa, Artificial Sequence
  Description of Artificial Sequence Synthetic peptide
ASTKGPSVFPLAP

SEQ ID NO: 30 - protein, 15 aa, Artificial Sequence
  Description of Artificial Sequence Synthetic peptide
GGGGSGGGGSGGGGS

SEQ ID NO: 31 - protein, 15 aa, Artificial Sequence
  Description of Artificial Sequence Synthetic peptide
GENKVEYAPALMALS

SEQ ID NO: 32 - protein, 15 aa, Artificial Sequence
  Description of Artificial Sequence Synthetic peptide
GPAKELTPLKEAKVS

SEQ ID NO: 33 - protein, 15 aa, Artificial Sequence
  Description of Artificial Sequence Synthetic peptide
GHEAAAVMQVQYPAS

SEQ ID NO: 34 - protein, 24 aa, Artificial Sequence
  Description of Artificial Sequence Synthetic peptide
TVAAPSVFIFPPTVAAPSVFIFPP

SEQ ID NO: 35 - protein, 26 aa, Artificial Sequence
  Description of Artificial Sequence Synthetic peptide
ASTKGPSVFPLAPASTKGPSVFPLAP

SEQ ID NO: 36 - protein, 5 aa, Artificial Sequence
  Description of Artificial Sequence Synthetic peptide
GGGGS

SEQ ID NO: 37 - protein, 7 aa, Artificial Sequence
  Description of Artificial Sequence Synthetic peptide
GGGGSGG

SEQ ID NO: 38 - protein, 10 aa, Artificial Sequence
  Description of Artificial Sequence Synthetic peptide
GGGGSGGGGS

SEQ ID NO: 39 - protein, 13 aa, Artificial Sequence
  Description of Artificial Sequence Synthetic peptide
GGGGSGGGGSGGG

SEQ ID NO: 40 - protein, 7 aa, Artificial Sequence
  Description of Artificial Sequence Synthetic peptide
TPAPLPT

SEQ ID NO: 41 - protein, 13 aa, Artificial Sequence
  Description of Artificial Sequence Synthetic peptide
TPAPLPAPLPAPT

SEQ ID NO: 42 - protein, 9 aa, Artificial Sequence
  Description of Artificial Sequence Synthetic peptide
TSPPSPAPE

SEQ ID NO: 43 - protein, 12 aa, Artificial Sequence
  Description of Artificial Sequence Synthetic peptide
TSPPSPAPELLG

SEQ ID NO: 44 - protein, 124 aa, Artificial Sequence
  Description of Artificial Sequence Synthetic polypeptide
EVQLVQSGAEVKKPGASVKVSCKASGYTFNNYGIIWVRQAPGQGLEWMGWINTYTGKPTYAQKFQGRVTMTTDTSTSTAYMELSSLRSEDTAVYYCARKLFNTVAVTDNAMDYWGQGTTVTVSS

SEQ ID NO: 45 - protein, 108 aa, Artificial Sequence
  Description of Artificial Sequence Synthetic polypeptide
DIQMTQSPSSLSASVGDRVTITCRASQDIENYLNWYQQKPGKAPKLLIYYTSRLQSGVPSRFSGSGSGTDFTLTISSLQPEDFATYFCQQGNTQPPTFGQGTKLEIKR

SEQ ID NO: 46 - protein, 117 aa, Artificial Sequence
  Description of Artificial Sequence Synthetic polypeptide
EVQLVESGGGLVQPGGSLRLSCAASGFTFSNYGVEWVRQAPGKGLEWVSGIWADGSTHYADTVKSRFTISRDNSKNTLYLQMNSLRAEDTAVYYCAREWQHGPVAYWGQGTLVTVSS

SEQ ID NO: 47 - protein, 108 aa, Artificial Sequence
  Description of Artificial Sequence Synthetic polypeptide
DIQMTQSPSSLSASVGDRVTITCKASQLVSSAVAWYQQKPGKAPKLLIYWASTLHTGVPSRFSGSGSGTDFTLTISSLQPEDFATYYCQQHYRTPFTFGQGTKLEIKR

SEQ ID NO: 48 - protein, 116 aa, Artificial Sequence
  Description of Artificial Sequence Synthetic polypeptide
EVQLVESGGGLVQPGGSLRLSCAASGFTFSNYGVTWVRQAPGKGLEWVSMIWADSTHYASSVKGRFTISRDNSKNTLYLQMNSLRAEDTAVYYCAREWQHGPVAYWGQGTLVTVSS

SEQ ID NO: 49 - protein, 108 aa, Artificial Sequence
  Description of Artificial Sequence Synthetic polypeptide
DIQMTQSPSSLSASVGDRVTITCRASQLVSSAVAWYQQKPGKAPKLLIYWASARHTGVPSRFSGSGSGTDFTLTISSLQPEDFATYYCQQHYKTPFTFGQGTKLEIKR

SEQ ID NO: 50 - protein, 121 aa, Artificial Sequence
  Description of Artificial Sequence Synthetic polypeptide
EVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 51 - protein, 108 aa, Artificial Sequence
  Description of Artificial Sequence Synthetic polypeptide
DIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 52 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFRLYSKLTVDKSRWQQGNVFSCSVMHEALHNHYTQKSLSLSPGK

SEQ ID NO: 53 - protein, 234 aa, Artificial Sequence
  Description of Artificial Sequence Synthetic polypeptide
VQCSGTTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 56 - protein, 106 aa, Artificial Sequence
  Description of Artificial Sequence Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 13 aa, Artificial Sequence
  Description of Artificial Sequence Synthetic peptide
ASTKGPSVFIFPP

SEQ ID NO: 58 - protein, 6 aa, Artificial Sequence
  Description of Artificial Sequence Synthetic peptide
ASTVAP

SEQ ID NO: 59 - protein, 12 aa, Artificial Sequence
  Description of Artificial Sequence Synthetic peptide
TVAAPSVFPLAP

SEQ ID NO: 60 - protein, 6 aa, Artificial Sequence
  Description of Artificial Sequence Synthetic peptide
TVASTP